(19) United States
(12) Patent Application Publication
(10) Pub. No.: US 2025/0388850 A1
(43) Pub. Date: Dec. 25, 2025

(54) ENHANCED TRIACYLGLYCEROL PRODUCTIVITY AND EXTRACTABILITY IN AN ENGINEERED MICROALGA

(71) Applicants: COMMISSARIAT A L'ENERGIE ATOMIQUE ET AUX ENERGIES ALTERNATIVES, PARIS (FR); UNIVERSITE GRENOBLE ALPES, SAINT-MARTIN-D’HERES (FR); CENTRE NATIONAL DE LA RECHERCHE SCIENTIFIQUE (CNRS), PARIS (FR); Institut national de recherche pour l’agriculture, l’alimentation et l’environnement, PARIS (FR)

(72) Inventors: Damien LE MOIGNE, GRENOBLE (FR); Juliette SALVAING, GRENOBLE CEDEX 09 (FR); Alberto AMATO, GRENOBLE (FR)

(21) Appl. No.: 18/881,376

(22) PCT Filed: Jul. 6, 2023
(86) PCT No.: PCT/EP2023/068788

§ 371 Date: Jan. 6, 2025

(30) Foreign Application Priority Data
Jul. 8, 2022 (EP) 22306024.5

(51) Int. Cl. C12N 1/12; C12N 9/22; C12N 15/113; C12N 15/79; C12P 7/6463; C12R 1/89

(52) U.S. Cl. CPC C12N 1/125; C12N 9/226; C12N 15/113; C12N 15/79; C12P 7/6463; C12N 2310/20; C12R 2001/89

(57) ABSTRACT

The present invention relates to an engineered unicellular Stramenopile microalga comprising a loss of function of the homologous Seipin gene, an in vitro method of producing triacylglycerols (TAG), and uses thereof.

### TECHNICAL FIELD OF THE INVENTION

The present invention relates to the production of oil by engineered microalgae, in particular the increased production of triacylglycerols (TAGs) by engineered diatom microalgae.

### BACKGROUND ART

The dependence on fossil fuels led to environmental problems and energy crises, so there is a continuous search for sustainable and renewable energy sources for producing biofuels in biological feedstock such as seed oils, animal fat, or oleaginous microorganisms. Microalgae are very promising organisms for the production of third generation biofuels. The use of microalgae allows to alleviate some of the drawbacks linked to the production of first and second generations biofuels. They can grow fast, use little water and do not compete with food crops for arable lands. Moreover, unlike other microorganisms studied for third generation biofuels, microalgae are autotrophic and consume CO₂; the marine species in particular are of interest as they do not use freshwater and can be grown using industrial waste. Finally, oil from microalgae can be enriched in omega-3 fatty acids, which makes them of interest for other applications, in particular food, feed and cosmetics. The review (Hu Qiang et al., 2008) presents the features of TAGs production by microalgae, notably the involved biosynthesis pathways and the parameters influencing the production (pH, temperature, light, . . . ).

Currently, two main locks that considerably increase the production costs, in particular compared to classical fuels, hinder the production of third generation biofuels. The first lock concerns the biomass production. Oil accumulation in microalgae is triggered by stress conditions that result in a slowing or even arrest of growth. Moreover, the maximal oil content is very dependent on the considered species. The second lock is linked to oil extraction, which can be broken down in two phases: 1) breaking the cells to free the oil and 2) separating the oil from the other cellular components. The actual methods used for oil extraction demand high energy and are thus very costly.

To bypass the first lock and trigger oil accumulation while maintaining cell growth, many studies have tried to modify the expression of enzymes involved in oil production or more broadly involved in lipid metabolism (review Kang et al. 2022). Some other studies have focused on proteins associated with Lipid Droplets (LD), the organelles in which oil is stored in cells. In parallel, efforts to bypass the second lock mostly regard development of new harvesting and lipid extraction techniques (for review Kowthaman et al. 2022), while to the knowledge of the Applicant, no research is done on the biological side.

So, there is still a need to provide new methods of increasing the triacylglycerol (TAGs) production and facilitate the extraction from the cell.

The Applicant surprisingly demonstrates that silencing the expression of the Seipin gene from the diatom Phaeodactylum tricornutum (PtSeipin gene) in the microalgae gives an increase of TAGs production without any negative impact on cell growth, whereas previous works made in yeasts and plants suggested an opposite effect on TAG production and/or cell growth.

Indeed, previous works on yeast and plants had shown that overexpressing Seipin proteins leads to higher TAG accumulation (Cai et al., 2015) and loss of Seipin triggers the formation of few very large LD instead of several small ones, with very little changes in oil accumulation (Fei et al., 2011; Taurino et al., 2018; WO 2012/075543).

So, the invention relates to an engineered Stramenopile microalga having a loss of function of the homologous Seipin gene, in particular in the homologous Seipin gene of Phaeodactylum tricornutum, leading to an increased production of triacylglycerol (TAG), a method of production of engineered microalga and uses thereof.

### SUMMARY OF THE INVENTION

A first subject-matter of the present invention is an engineered unicellular Stramenopile microalga comprising a loss of function of the homologous Seipin gene.

The present invention also relates to a microalgae culture comprising an engineered unicellular Stramenopile microalga according to the invention, preferably an engineered unicellular Phaeodactylum tricornutum (from wild-type strain CCMP2561).

Another subject-matter of the present invention is a vector comprising Cas9 sequence and one single guide sgRNA designed to target a sequence upstream or downstream of the 1^{st} transmembrane domain, in particular one single guide sRNA 1 (SEQ ID NO. 24: AGAAGAAGCGCACGCTGCCG) or sgRNA 8 (SEQ ID NO. 25: TTCAATCCATACCGAGAGCA).

The present invention also relates to an in vitro method of producing triacylglycerols (TAG) comprising culturing an engineered microalga according to the invention or a microalgae culture of the present invention in a culture medium to produce TAG, in particular in normal growth conditions or preferably in stress conditions selected from nutrient starvation and/or light stress conditions, and optionally further comprising a step of recovering the TAG from the engineered microalgae, the culture medium or the whole culture.

Another subject-matter of the invention is a use of the engineered Stramenopile microalga according to the invention, or the microalgae culture of the invention, or directly the TAGs produced by the in vitro method according to the invention, for biofuel production, in food industry, in feed industry, in green chemistry, in pharmaceutical industry or for the production of cosmetics, in particular for biofuel production.

### DESCRIPTION OF THE FIGURES

FIG. 1: The evolutionary history was inferred using the Neighbor-Joining method (Saitou N. and Nei M, 1987). The optimal tree with the sum of branch length=48.43171304 is shown. The percentage of replicate trees in which the associated taxa clustered together in the bootstrap test (5000 replicates) are shown next to the branches. The tree is drawn to scale, with branch lengths in the same units as those of the evolutionary distances used to infer the phylogenetic tree. The evolutionary distances were computed using the JTT matrix-based method (Jones D. T. et al., 1992) and are in the units of the number of amino acid substitutions per site. The rate variation among sites was modeled with a gamma distribution (shape parameter=2). This analysis involved 52 amino acid sequences. All ambiguous positions were removed for each sequence pair (pairwise deletion option). There were a total of 1511 positions in the final dataset. Evolutionary analyses were conducted in MEGA X. This figure also illustrates the percentage identity of each Seipin sequence with Pt Seipin sequence, based on a reference sequence (SEQ ID NO: 23).

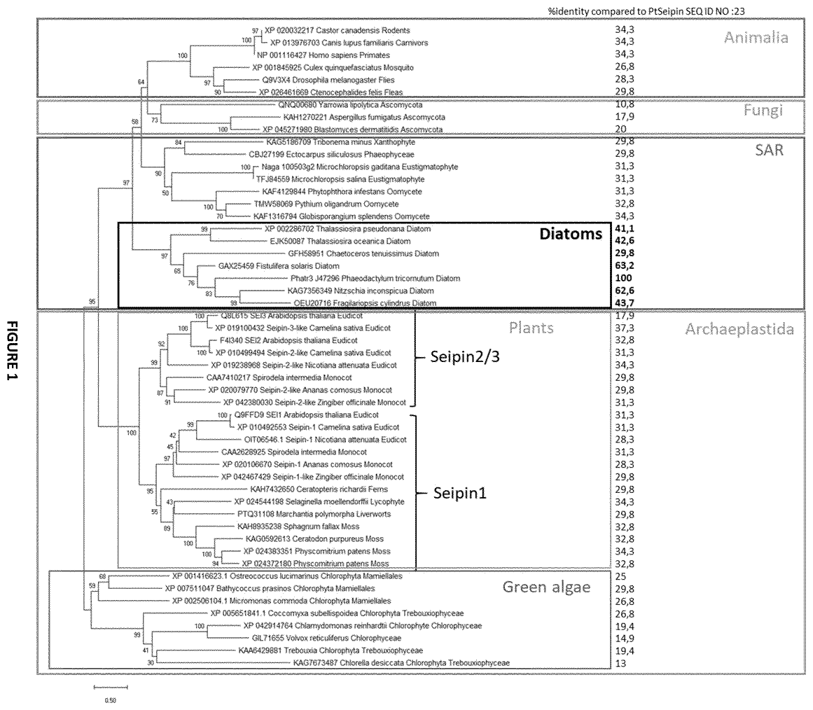

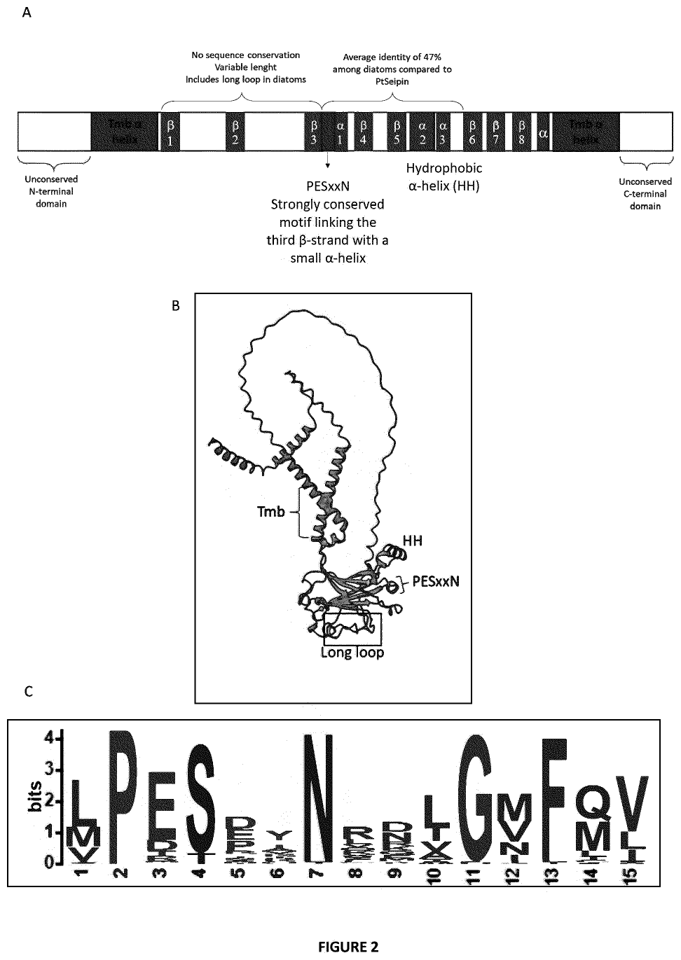

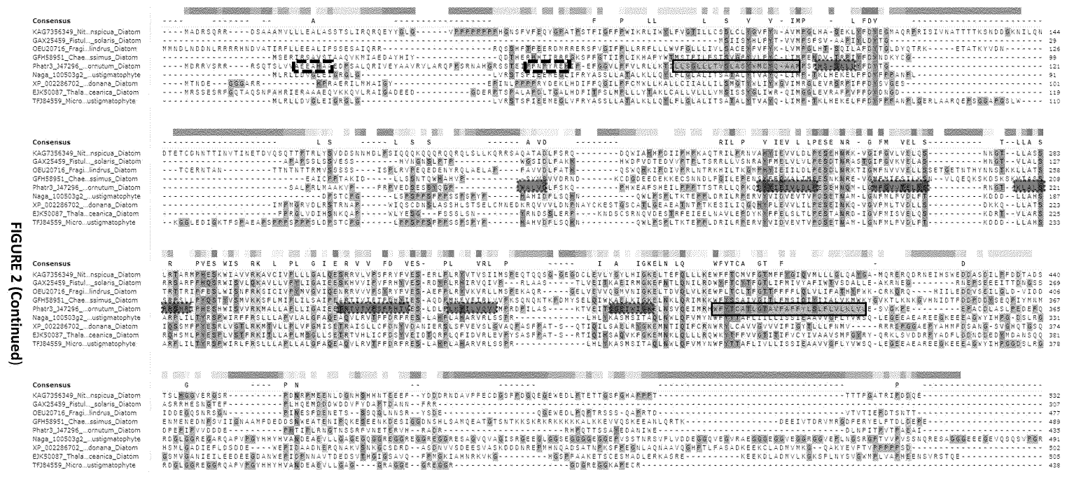

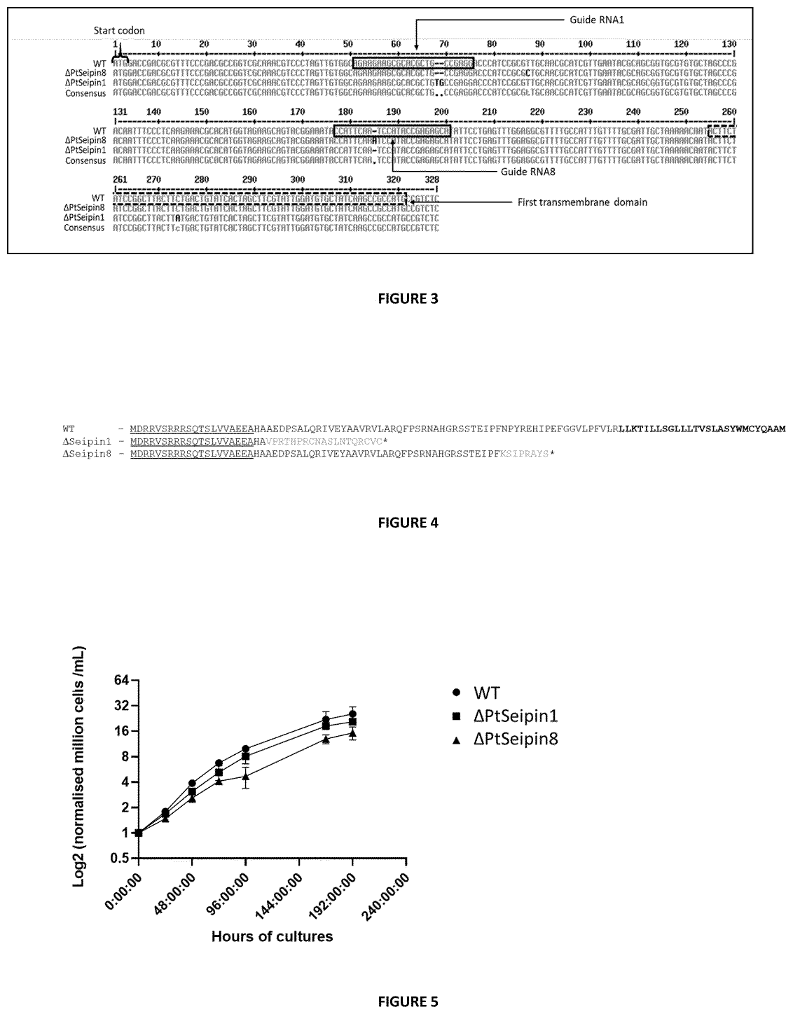

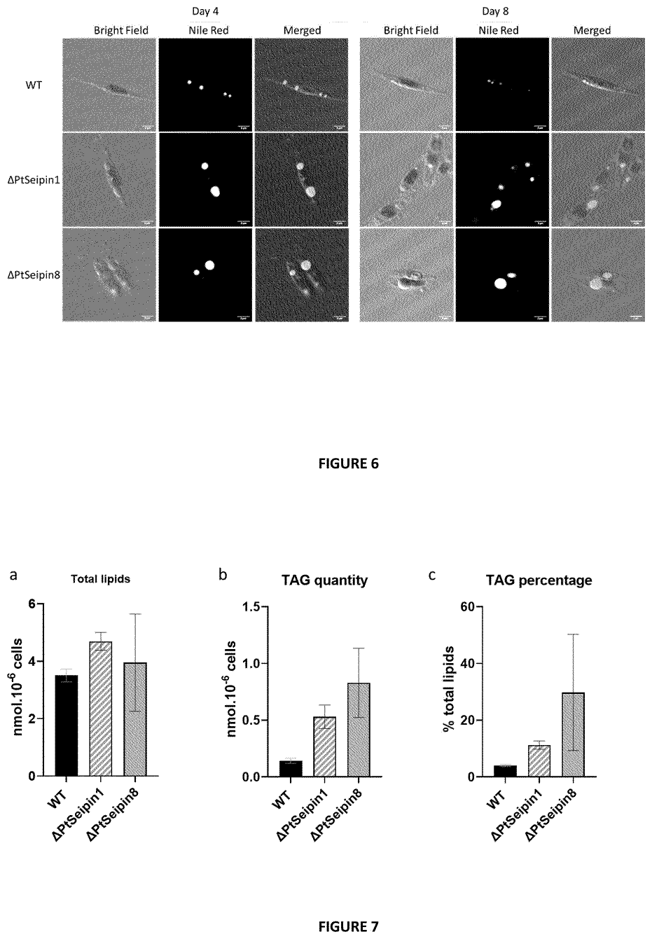

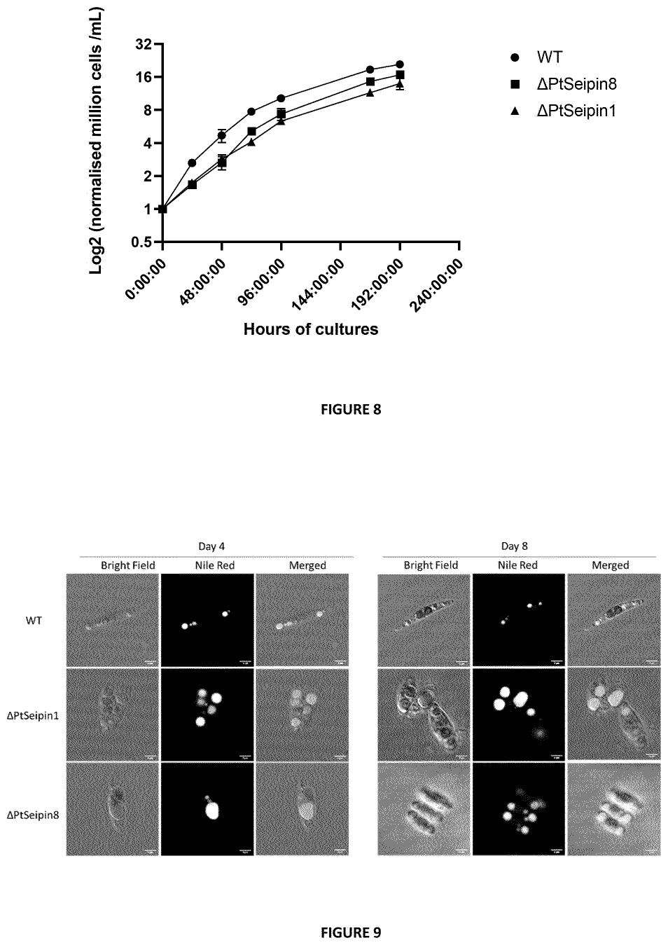

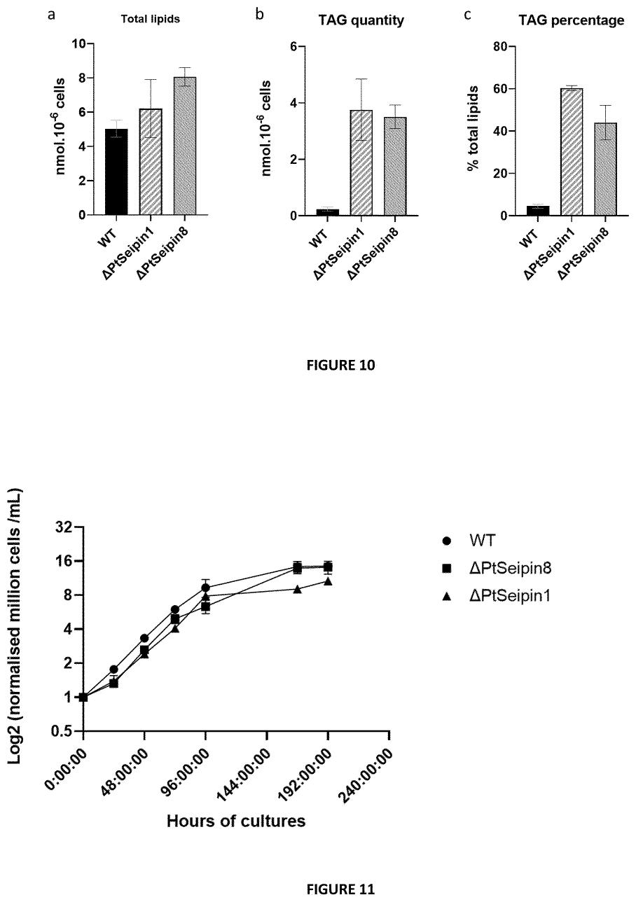

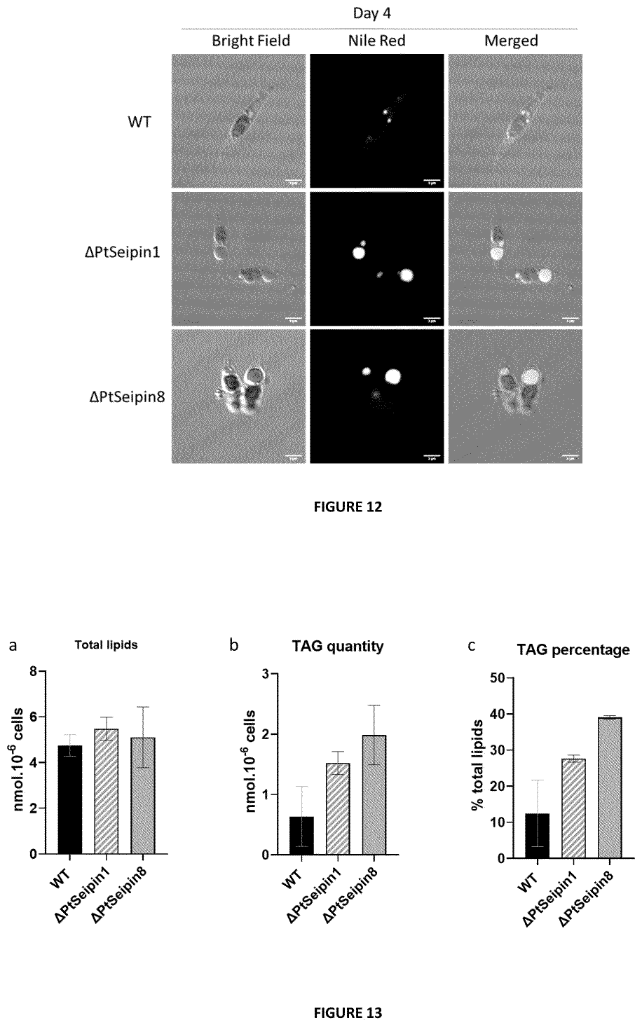

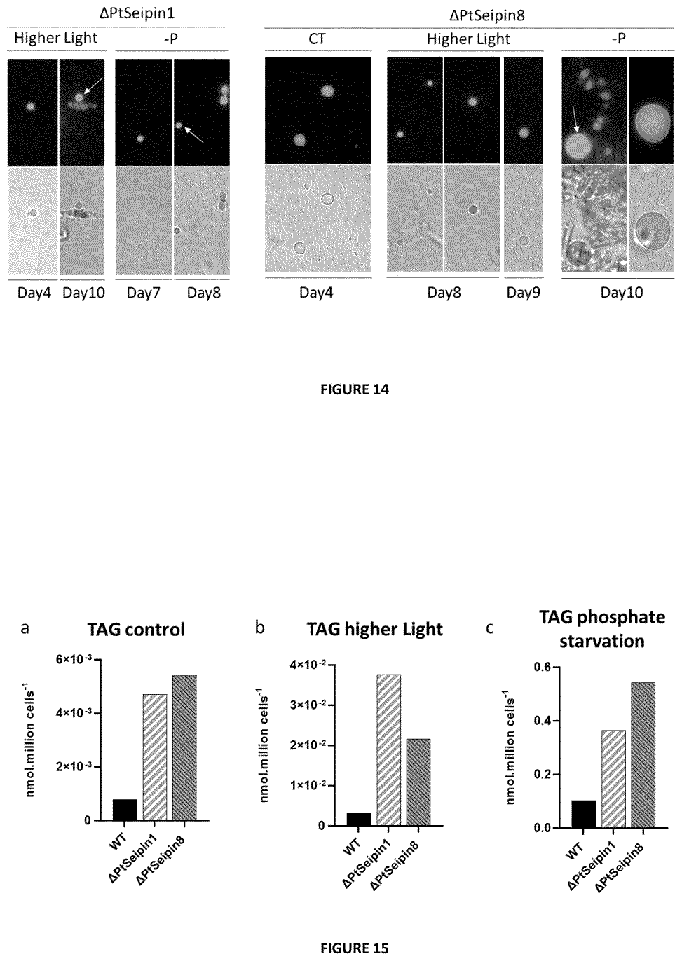

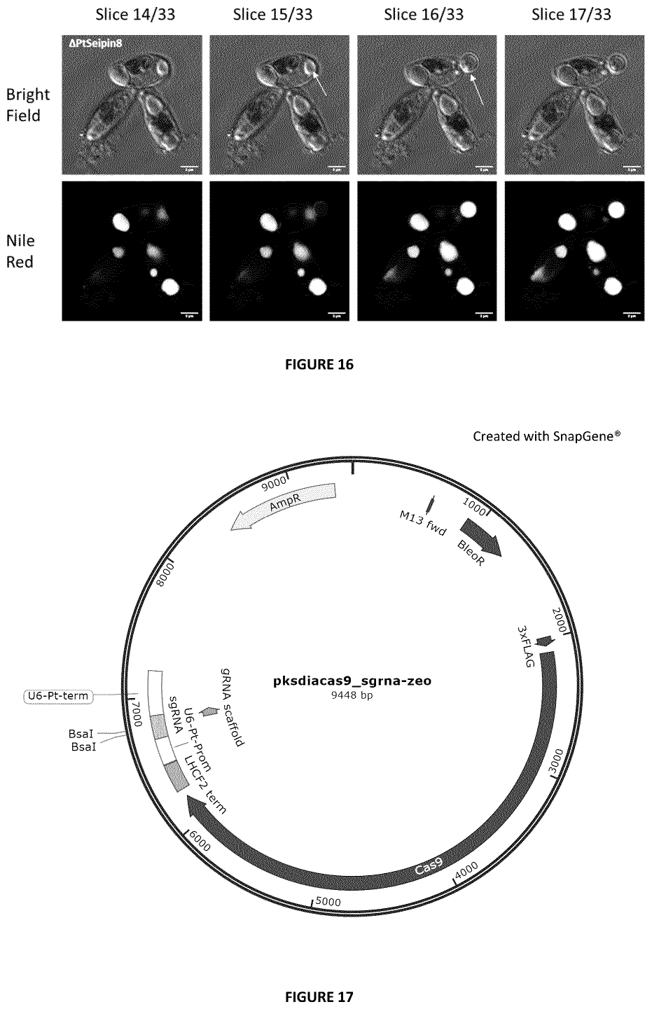

FIG. 2: Structural analysis of Seipins. A: cartoon representation showing the general organization common to most known Seipin proteins. The N- and C-terminal parts are very variable in length and structure and do not show any sequence conservation across phylla. The transmembrane domains form α-helixes but show very little sequence conservation. The central part is mainly composed by 8 β-strands (β1 to 8), forming a beta-sandwich. In human, this structure mediates the interaction with anionic lipids (Yan et al. 2018). The hydrophobic α-helix (α2-α3), located between the 5^{th} and the 6^{th} β-strands, plays an important role in Seipin oligomerization (Sui et al. 2018, Yan et al. 2018) as well as TAG clustering (Zoni et al., 2021). Its structure and localization are highly conserved yet it shows only limited sequence conservation. Another very small α-helix (α1) is found between the 3^{rd} and the 4^{th} β-strands in association with a very conserved small motif (PESxxN); yet its function has not been investigated. B: AlphaFold prediction of PtSeipin structure. The main features are highlighted, including the long loop found in diatoms between the 1^{st} and the 3^{rd} β-strands, between amino acids positions P107 and L192 included, in the sequence of PtSeipin. Tmb: transmembrane domain. C: The PESxxN motif as identified in all of the 52 sequences used for the construction of the phylogenetic tree (FIG. 1) by the MEME tool.
D: Alignment of amino acid sequences of seven Seipin proteins from diatoms microalgae, respectively: Nitzschia inconspicua (accession number KAG7356349), Fistulifera solaris (accession number GAX25459), Fragilariopsis cylindrus (accession number OEU20716), Chaetoceros tenuissimus (accession number GFH58951), Phaeodactylum tricornutum (accession number Phatr3_J47296), Thalassiosira pseudonana (accession number XP_002286702), and Thalassiosira oceanica (accession number EJK50087) as well as 2 amino acid sequences of two Seipin proteins from Eustigmatophytes, respectively Microchloropsis gaditana (accession number Naga_100503g2) and Microchloropsis salina (accession number TFJ84559). Legend: large dotted lines: position of sgRNA 1 and 8; Full dotted lines: transmembrane domain; Small dotted lines: b-strand.

FIG. 3: Nucleotidic alignement of the first part of the PtSeipin gene obtained from sequencing of the WT and ΔPtSeipin lines. Alignments were realised using the Multalin software (http://multalin.toulouse.inra.fr/multalin/). The sequences targeted by the two guide RNAs (guide RNA1 and guide RNA8) are boxed in solid line. Both are upstream of the sequence of the first transmembrane domain, which is boxed with a dotted line.
WT: SEQ ID NO. 26; ΔPtSeipin8: SEQ ID NO. 27; ΔPtSeipin1: SEQ ID NO. 28; Consensus: SEQ ID NO. 29.

FIG. 4: Protein translation of Phaeodactylum tricornutum Seipin gene in WT (incomplete) and mutants. The underlined part corresponds to the signal peptide and the bolded part in the WT corresponds to the first transmembrane domain. The parts in light grey in the mutant sequences correspond to parts between the frameshift and the end of translation where the translated sequence is different from the WT sequence. *: stop codon. WT: SEQ ID NO. 30; ΔSeipin1: SEQ ID NO. 21; ΔSeipin8: SEQ ID NO. 22.

FIG. 5: Growth curves of the Wild Type (WT) and knock-out (ΔPtSeipin1 and ΔPtSeipin8) lines in control condition (ESAW 10N10P, 75 μmol·photon·m²·s⁻¹, 20° C., 100 rpm). Absorbance at 730 nm is used to measure cell concentration. Cell concentrations are normalized for each time point by the initial concentration (T0). Log base 2 scale is used to reflect cell divisions (biomass duplicates every 24 hours in optimal conditions). Cultures were followed for 8 days.

FIG. 6: Lipid droplets accumulation in control condition after 4 and 8 days of cultures. Images were acquired by confocal microscopy after Nile Red staining. Bright Field, Nile red fluorescence (excitation: 514 nm-emission: 580-640 nm) and merged signal representative images are shown. Scale bar=3 μm.

FIG. 7: Oil (TAG) accumulation in control (normal growth) condition in WT and mutants after 4 days of cultivations (end of exponential phase). (a): total amount of lipids in nmol per million cells. (b): Triacylglycerols (TAG) quantity in nmol per million cells increases by a 3.8 (mutant ΔPtSeipin1) to 5.8 (mutant ΔPtSeipin8) fold ratio. (c): Triacylglycerols (TAG) in percentage of total glycerolipids increase by 2.78 (mutant ΔPtSeipin1) and 7.4 (mutant ΔPtSeipin8) fold ratio compared to the WT.

FIG. 8: Growth curves of the knock-out (ΔPtSeipin1 and ΔPtSeipin8) lines and WT in the higher light condition (ESAW 10N10P, 200 μmol·photon·m² s⁻¹, 20° C., 100 rpm). Absorbance at 730 nm is used to measure cell concentration. Cell concentrations were normalized for each time point by the initial concentration (T0). Log base 2 scale is used to reflect cell divisions (biomass duplicates every 24 hours in optimal conditions). Cultures were followed for 8 days.

FIG. 9: Lipid droplets accumulation in higher light condition after 4 and 8 days of cultures. Images were acquired by confocal microscopy after Nile Red staining. Bright Field, Nile red fluorescence (excitation: 514 nm—emission: 580-640 nm) and merged signal representative images are shown. Scale bar=3 μm.

FIG. 10: Oil (TAG) accumulation in WT and mutants after 8 days of exposure to higher light (200 μmol·photon·m²·s⁻¹). (a): total amount of lipids in nmol per million cells. (b): Triacylglycerols (TAG) quantity in nmol per million cells increases by a 16.3 (ΔPtSeipin1) and 15.2 (ΔPtSeipin8) fold ratio. (c): Triacylglycerols (TAG) in percentage of total glycerolipids increase by a 13.3 fold ratio compared to the WT for ΔPtSeipin1 and 9.7 fold ratio for ΔPtSeipin8.

FIG. 11: Growth curves of the Wild Type (WT) and knock-out (ΔPtSeipin1 and ΔPtSeipin8) lines in phosphate starvation condition (ESAW 10N00P, 75 μmol·photon·m²·s⁻¹, 20° C., 100 rpm). Absorbance at 730 nm is used to measure cell concentration. Cell concentrations were normalized for each time point by the initial concentration (T0). Log base 2 scale is used to reflect cell divisions (biomass duplicates every 24 hours in optimal conditions). Cultures were followed for 8 days.

FIG. 12: Lipid droplets accumulation in phosphate starvation condition after 4 days of cultures. Images were acquired by confocal microscopy after Nile Red staining. Bright Field, Nile red fluorescence (excitation: 514 nm—emission: 580-640 nm) and merged signal representative images are shown. Scale bar=3 μm.

FIG. 13: Oil (TAG) accumulation in phosphate starvation condition after 4 days of cultures (a): total amount of lipids per million cells. (b): Triacylglycerols (TAG) quantity per million cells increases by a 2.4 (ΔPtSeipin1) and 3.1 (ΔPtSeipin8) fold ratio. (c): Similar fold change in TAG accumulation are observed in percentage of total glycerolipids: 2.2 fold ratio between WT for ΔPtSeipin1 and 3.1 fold ratio for ΔPtSeipin8.

FIG. 14: Epifluoresence images of Lipid droplets floating in the culture medium. Left part: images on mutant ΔPtSeipin1. Right part: images on mutant ΔPtSeipin8. White arrows indicate the LDs on the images where a microalga is also present CT: Control condition, Higher Light: higher light condition, —P: phosphate deficiency condition.

FIG. 15: TAG accumulation in the culture medium of Wild Type (WT) and knock-out (ΔPtSeipin1 and ΔPtSeipin8) lines (a): Control condition (ESAW 10N10P, 75 μmol·photon·m²·s⁻¹, 20° C., 100 rpm). (b): Higher light condition (ESAW 10N10P, 200 μmol·photon·m²·s⁻¹, 20° C., 100 rpm). (c): Phosphate starvation condition (ESAW 10N00P, 75 μmol·photon·m²·s⁻¹, 20° C., 100 rpm). The TAG quantity measured in nmol is normalized by the cell number in the total culture.

FIG. 16: Confocal microscope z-stack acquisition on the ΔPtSeipin8 mutant after 4 days of exposure to higher light. Bright field and Nile red signals are shown. Four slices from the z stack acquisition (33 slices in total) are shown. The Lipid droplet liberation can be observed between slice 15 and slice 16 acquisition and is highlighted by white arrows. Scale bar=3 μm.

FIG. 17: Vector map of the pKSdiaCAs9_sgRNA_ZeoR plasmid (Seydoux et al. 2022) used in the examples.

### DETAILED DESCRIPTION OF THE INVENTION

A first object of the invention is an engineered unicellular Stramenopile microalga comprising a loss of function of the homologous Seipin gene.

In other words, the present invention concerns an engineered unicellular Stramenopile microalga, wherein the Seipin gene from Phaeodactylum tricornutum having the sequence SEQ ID NO. 3, or one of its homologs, is silenced.

### Definitions

The terms “engineered” as used herein with reference to a Stramenopile microalga, defines a non-naturally occurring microalga, as well as its recombinant progeny, that has at least one genetic alteration not found in a naturally occurring microalga, including wild-type microalga of the same type. Such genetic modification is typically achieved by technical means (i.e. non-naturally) through human intervention and may include, e.g., the introduction of an exogenous nucleic acid and/or the modification or deletion of an endogenous nucleic acid.

As used herein, the expression “microalgae” refers to microscopic algae, with sizes from a few micrometers to a few hundred micrometers.

The microalgae of interest in the present invention for the production of TAG are algae belonging to the Stramenopiles, (also named Heterokont phylum or Heterokonts) which include the classes Bacillariophycea (diatoms), Eustigmatophycea, Phaeophyceae (brown algae), Xanthophyceae (yellow-green algae) and Chrysophyceae (golden algae). The invention mainly focuses on Stramenopiles.

Diatom is a major group of unicellular photosynthetic heterokonts (or stramenopiles) microalgae, living in oceans and freshwaters.

They are found in diverse environments, in aquatic and soil ecosystems, and are major contributors to the ocean's carbon, nitrogen and silicon cycles. The oleaginous marine diatom Phaeodactylum tricornutum has a fully sequenced and annotated genome (Bowler et al., 2008; accession number GCA_000150955).

In particular, the microalgae with high industrial potential (for example used as food supplements or used for biofuel production) are Phaeodactylum tricornutum and Thalassiosira pseudonana, preferably Phaeodactylum tricornutum.

A “homologous gene” is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor.

The term “homolog” as used herein in connection to Phaeodactylum tricornutum (Pt) Seipin gene refers to the fact that the homologous gene differs from Pt Seipin gene having the sequence SEQ ID NO. 3 in its sequence, but that retains the activity of Pt Seipin protein, and originates from another species, i.e. is a naturally occurring sequence. A homolog of Pt Seipin can be identified by the skilled person by pairwise search methods such as BLAST and checking of the corresponding activity.

In particular, mention may be made of diatoms Seipin of Nitzschia inconspicua (accession number KAG7356349), Fistulifera solaris (accession number GAX25459), Fragilariopsis cylindrus (accession number OEU20716), Chaetoceros tenuissimus (accession number GFH58951), Phaeodactylum tricornutum (accession number Phatr3_J47296), Thalassiosira pseudonana (accession number XP_002286702), and Thalassiosira oceanica (accession number EJK50087).

The article (Gueguen et al., 2021) reports identification of Seipin homologs in microalgae by sequence homology: with this method, no homolog has been identified in Nannochloropsis species, since sequence of this protein is poorly conserved across species. Later, with the help of structure predictions, in particular the AlphaFold protein structure database, Seipin homologs have been identified in Michrochloropsis gaditana (UNIPROT accession number W7TBE7) and Microchloropsis salina (UNIPROT accession number A0A4D9D7J7). These proteins present a high homology of structure, but not of sequence, and in particular comprise a long loop between the 1st and the third β-strands of the beta-sandwich of the lumenal domain, with a length of at least 80 amino acids located between the end of the 1st transmembrane domain and the conserved motif PESxxN (or PDSxxN) located at the end of the third β-strand.

By ‘loss of function of Seipin gene’, it means that the activity of the targeted Seipin gene is reduced or abolished. Different mechanisms are known for silencing gene expression. Mention may be made to mutation, RNA interference, antisens DNA, Knock-out, or small molecules inhibitors.

A “mutation” as used herein, refers to a change in nucleic acid sequence relative to a reference Seipin gene sequence (which is preferably a naturally-occurring normal or «wild-type» sequence), and includes translocations, deletions, insertions, and substitutions mutations.

Suitable genetic engineering methods for introducing a mutation in an endogenous gene are known to the skilled person, including by using so-called molecular scissors (nucleases) (e.g. TALEN, CRISPR/Cas9 and the like), or by using vectors containing specific sequences for homologous recombination and site-directed insertion.

The term “mutant Seipin”, as used in the present invention refers to a Seipin protein comprising in its amino acid sequence one or more additions, deletions and/or substitutions. In a particular embodiment, the mutant Seipin is a truncated non-functional protein, wherein sequence mutations lead to the introduction of premature stop codons.

As used herein, “triacylglycerols” or “triacylglycerides” (TAG) are esters resulting from the esterification of the three hydroxyl groups of glycerol, with three fatty acids.

In a triacylglyceride, the glycerol may be linked to saturated and/or unsaturated fatty acids. The triacylglycerides produced in the invention preferably contain one, two or three saturated and/or monounsaturated fatty acids. More preferred are triacylglycerides containing one, two or three saturated fatty acids. In particular, TAGs produced by Phaeodactylum tricornutum comprise palmitic acid (C16:0) and palmitoleic acid (C16:1).

The table 1 hereunder discloses the several sequences illustrated in the examples of the present invention, but the invention is not limited to said sequences.

TABLE 1

| | | |
|---|---|---|
| SEQ ID | | |
| NO: | Type of sequence | Sequence |

SEQUENCES OF Phaeodactylum tricornutum (Pt) Seipin (strain CCAP 1055/1,
Phatr3_J47296)

| | | |
|---|---|---|
| 1 | Amino acid sequence of the | 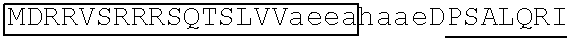 |
| | Phaeodactylum tricornutum | VEYAAVRVLARQFPSRNAHGRSSTEIpfnpyre |
| | (Pt) Seipin (strain CCAP | hIPEFGGVLPFVLRLLKTILLSGLLLTVSLASY |
| | 1055/1, Phatr3_J47296) with | WMCYQAAM[PSRHSSKLLYFDYTGSALPRL |
| | annotations of secondary | MAAKVPFRPVEDSESSYQGPWALVDLFSKQ |
| | structures as predicted by | PHWEAFSHEILPPPTTSTRLLQPKQDYYIEIVL |
| | alpha-fold | DL]PESEHNQMLGMFGVVTELYSRNGTKLAL |
| | | SRRSMRIPHESHWISVVRKMALLAPLLIGAIE |
| | In framed characters: predicted | ETRTVTVPSFRHIVESLDLPLKYILVQVVMPR |
| | signal peptide; underlined: alpha | DPILASKTVEITGGEIWIGKELNSVQEIMRHW |
| | helices; in italic characters: beta | FYTCATLGTAVFAFFYLSLFLVLKLVLESVGK |
| | strands; in bold characters: | PEEPACDLASLPEDEPQDFEPIPVVDDDEPH |
| | transmembrane domains; | TIPLRNGTNSSRFVNETERVHRADRRHPQTT |
| | highlighted in lower cases | SSAYEDANIWEDLNFITPVTPAEAI |
| | characters: areas coded by the | |
| | sequences corresponding to the | |
| | sgRNAs illustrated in the examples. | |
| | Sequence inside brackets: long loop | |
| | specific of diatoms sequences; | |
| | comprises 86 amino acids in Pt | |
| | Seipin between the 1st | |
| | transmembrane domain and the | |
| | PESxxN motif (between amino acids | |
| | positions P107 and L192 included, in | |
| | the sequence of PtSeipin). | |
| 2 | Genomic sequence of the | cgattttcgaatctggaacgaacgaaacaaacctgccacacc |
| | Phaeodactylum tricornutum | atgagaatgattgaaatgagcaacagcgcttctcggaagaaa |
| | (Pt) Seipin gene (strain CCAP | ttgtgtgattagagggtaacaaggaacccttagacataacagt |
| | 1055/1, Phatr3_J47296) with | cagccggagctctttagaatcggtgtcctgtcagcaggaagcg |
| | annotations | acttcaacacctcgccgttcaagaatcagaaaggATGGAC |
| | | CGACGCGTTTCCCGACGCCGGTCGCAAAC |
| | In lower case characters: 5'-UTR; in | GTCCCTAGTTGTGGCAGAAGAAGCGCACG |
| | capital letters: the two exons, and in | CTGCCGAGGACCCATCCGCGTTGCAACGC |
| | capital and italic, bold characters: the | ATCGTTGAATACGCAGCGGTGCGTGTGCT |
| | intron. The sequences | AGCCCGACAATTTCCCTCAAGAAACGCACA |
| | corresponding to guide RNAs 1 (on | TGGTAGAAGCAGTACGGAAATACCATTCAA |
| | the sense strand) and 8 (on the | TCCATACCGAGAGCATATTCCTGAGTTTGG |
| | antisense strand) as illustrated in the | AGGCGTTTTGCCATTTGTTTTGCGATTGCT |
| | examples, are underlined and the | AAAAACAATACTTCTATCCGGCTTACTTCTG |
| | NGG of said underlined sequences | ACTGTATCACTAGCTTCGTATTGGATGTGC |
| | is in bold characters. | TATCAAGCCGCCATGCCGTCTCGGCACTC |
| | | CAGTAAGCTGCTGTATTTCGACTATACAGG |
| | | GTCTGCGTTGCCACGGCTGATGGCAGCCA |
| | | AAGTTCCATTTCGCCCCGTCGAAGACAGC |
| | | GAGTCCAGCTATCAAGGTCCATGGGCTCT |
| | | GGTAGATTTGTTCTCTAAACAGCCGCACTG |
| | | GGAGGCATTCAGTCATGAAATTCTACCGCC |
| | | GCCTACCACATCGACGCGACTTTTACAGCC |
| | | CAAGCAGGACTACTACATTGAAATAGTGTT |
| | | GGACTTACCGGAGTCGGAACACAATCAGA |
| | | TGCTGGGCATGTTTGGTGTCGTCACGGAA |
| | | CTCTATTCACGCAACGGAACCAAACTTGCC |
| | | TTATCTCGGCGGTCGATGCGCATACCGCAT |
| | | GAGAGCCACTGGATATCGGTCGTGCGCAA |
| | | GATGGCACTCTTGGCACCCCTATTGATTGG |
| | | CGCCATCGAAGAAACACGAACGGTAACGG |
| | | TGCCATCATTTCGACACATTGTGGAAAGTC |
| | | TGGATTTACCACTGGTACGTTTCAATGGCC |
| | | AAGTTCTGTAATGACAGGCAGTGAATATT |
| | | ACCTTCTTTACTGACTAAACTGATTTTTGA |
| | | TCACACAGAAGTATATATTGGTCCAAGTCG |
| | | TCATGCCAAGAGACCCAATTCTGGCGTCGA |
| | | AAACAGTAGAGATCACTGGCGGTGAAATAT |
| | | GGATTGGCAAAGAGCTCAACAGCGTGCAG |
| | | GAAATTATGCGTCACTGGTTTTATACGTGT |
| | | GCTACCCTTGGTACTGCTGTCTTCGCTTTC |
| | | TTTTACTTGTCGTTGTTCCTGGTCCTAAAAC |
| | | TCGTCCTCGAATCTGTAGGCAAGCCGGAA |
| | | GAACCGGCATGCGACTTGGCAAGTTTGCC |
| | | TGAGGATGAGCCGCAAGACTTTGAGCCCA |
| | | TCCCAGTGGTAGATGACGATGAGCCACATA |
| | | CTATCCCGTTACGCAACGGAACGAATAGCT |
| | | CGCGGTTTGTGAATGAAACGGAGCGTGTC |
| | | CACCGTGCCGATCGGCGACATCCACAGAC |
| | | TACATCATCTGCTTACGAAGACGCAAATAT |
| | | TTGGGAAGACTTGAATTTTATTACTCCGGTT |
| | | ACTCCAGCCGAAGCTATTTGA |
| 3 | Coding nucleic acid sequence | ATGGACCGACGCGTTTCCCGACGCCGGTC |
| | of the Phaeodactylum | GCAAACGTCCCTAGTTGTGGCAGAAGAAG |
| | tricornutum (Pt) Seipin gene | CGCACGCTGCCGAGGACCCATCCGCGTTG |
| | (strain CCAP 1055/1, | CAACGCATCGTTGAATACGCAGCGGTGCG |
| | Phatr3_J47296) | TGTGCTAGCCCGACAATTTCCCTCAAGAAA |
| | Same annotations as for the above | CGCACATGGTAGAAGCAGTACGGAAATAC |
| | genomic sequence, except that the | CATTCAATCCATACCGAGAGCATATTCCTG |
| | 5'UTR is lacking | AGTTTGGAGGCGTTTTGCCATTTGTTTTGC |
| | ATG: start codon | GATTGCTAAAAACAATACTTCTATCCGGCTT |
| | | ACTTCTGACTGTATCACTAGCTTCGTATTG |
| | | GATGTGCTATCAAGCCGCCATGCCGTCTC |
| | | GGCACTCCAGTAAGCTGCTGTATTTCGACT |
| | | ATACAGGGTCTGCGTTGCCACGGCTGATG |
| | | GCAGCCAAAGTTCCATTTCGCCCCGTCGAA |
| | | GACAGCGAGTCCAGCTATCAAGGTCCATG |
| | | GGCTCTGGTAGATTTGTTCTCTAAACAGCC |
| | | GCACTGGGAGGCATTCAGTCATGAAATTCT |
| | | ACCGCCGCCTACCACATCGACGCGACTTTT |
| | | ACAGCCCAAGCAGGACTACTACATTGAAAT |
| | | AGTGTTGGACTTACCGGAGTCGGAACACA |
| | | ATCAGATGCTGGGCATGTTTGGTGTCGTCA |
| | | CGGAACTCTATTCACGCAACGGAACCAAAC |
| | | TTGCCTTATCTCGGCGGTCGATGCGCATAC |
| | | CGCATGAGAGCCACTGGATATCGGTCGTG |
| | | CGCAAGATGGCACTCTTGGCACCCCTATTG |
| | | ATTGGCGCCATCGAAGAAACACGAACGGT |
| | | AACGGTGCCATCATTTCGACACATTGTGGA |
| | | AAGTCTGGATTTACCACTGGTACGTTTCAA |
| | | TGGCCAAGTTCTGTAATGACAGGCAGTGA |
| | | ATATTACCTTCTTTACTGACTAAACTGATTT |
| | | TTGATCACACAGAAGTATATATTGGTCCAA |
| | | GTCGTCATGCCAAGAGACCCAATTCTGGC |
| | | GTCGAAAACAGTAGAGATCACTGGCGGTG |
| | | AAATATGGATTGGCAAAGAGCTCAACAGCG |
| | | TGCAGGAAATTATGCGTCACTGGTTTTATA |
| | | CGTGTGCTACCCTTGGTACTGCTGTCTTCG |
| | | CTTTCTTTTACTTGTCGTTGTTCCTGGTCCT |
| | | AAAACTCGTCCTCGAATCTGTAGGCAAGCC |
| | | GGAAGAACCGGCATGCGACTTGGCAAGTT |
| | | TGCCTGAGGATGAGCCGCAAGACTTTGAG |
| | | CCCATCCCAGTGGTAGATGACGATGAGCC |
| | | ACATACTATCCCGTTACGCAACGGAACGAA |
| | | TAGCTCGCGGTTTGTGAATGAAACGGAGC |
| | | GTGTCCACCGTGCCGATCGGCGACATCCA |
| | | CAGACTACATCATCTGCTTACGAAGACGCA |
| | | AATATTTGGGAAGACTTGAATTTTATTACTC |
| | | CGGTTACTCCAGCCGAAGCTATTTGA |
| 4 | Targeted nucleic acid | AGAAGAAGCGCACGCTGCCGAGG |
| | sequence for sgRNA1 (on the | |
| | sense strand) with NGG in | |
| | bold character | |
| 5 | Targeted nucleic acid | CCATTCAATCCATACCGAGAGCA |
| | sequence for sgRNA8 (on the | |
| | antisense strand) with NGG in | |
| | bold character | |
| 6 | Long loop comprising the 1st | PSRHSSKLLYFDYTGSALPRLMAAKVPFRPV |
| | beta-strand and going up to | EDSESSYQGPWALVDLFSKQPHWEAFSHEIL |
| | the third ß-strand (included) | PPPTTSTRLLQPKQDYYIEIVLDL |
| | of the beta-sandwich of the | |
| | lumenal domain of | |
| | Phaeodactylum tricornutum | |
| | (Pt) Seipin (between amino | |
| | acids positions P107 and | |
| | L192 included, in the | |
| | sequence of PtSeipin) | |
| 7 | Amino acid sequence | IPHESHWISVVRKMALLAPLLIGAIEE |
| | between the 5th and 6th beta- | |
| | strands including the alpha- | |
| | hydrophobic helix | |
| | (underlined) that is conserved | |
| | between Diatoms Seipin | |
| | sequences | |
| | In bold characters: these amino acid | |
| | are not conserved in | |
| | Eustigmatophytes; they are | |
| | respectively R, R and S | |
| 8 | Amino acid pattern after the | PHES |
| | 5th beta-strand that is | |
| | conserved between the | |
| | Diatoms Seipin sequences | |
| | and not conserved in the | |
| | Eustigmatophytes Seipin | |
| | sequences | |
| 9 | Amino acid pattern in the 8th | IGKE |
| | beta-strand that is conserved | |
| | between the Diatoms Seipin | |
| | sequences and not conserved | |
| | in the Eustigmatophytes | |
| | Seipin sequences | |

PRIMERS

| | | |
|---|---|---|
| 10 | Seipin-g1-Fwd primer | TCGAAGAAGAAGCGCACGCTGCCG |
| | use for guide RNA1 | |
| | in bold characters: reconstitution of | |
| | the Bsal restriction site for cloning of | |
| | the guide RNA sequence in the | |
| | vector pKsDiaCas9_sgRNA (figure | |
| | 17) | |
| 11 | Seipin-g1-Rev primer | AAACCGGCAGCGTGCGCTTCTTCT |
| | use for guide RNA1 | |
| | in bold characters: same as above | |
| 12 | Seipin-g8-Fwd primer | TCGATGCTCTCGGTATGGATTGAA |
| | use for guide RNA8 | |
| | in bold characters: same as above | |
| 13 | Seipin-g8-Rev primer | AAACTTCAATCCATACCGAGAGCA |
| | use for guide RNA8 | |
| | in bold characters: same as above | |
| 14 | pCas9-U6-Rev primer | GACATGCTACCCGCAGACCG |
| | use for plasmid checking | |
| 15 | Seipin-PCR-Fwd primer | GGGGTTCCAAACATTCTGCC |
| | use for mutants screening - | |
| | PCR | |
| 16 | Seipin-PCR-Rev primer | CACAAACCGCGAGCTATTCG |
| | use for mutants screening - | |
| | PCR | |
| 17 | Seipinseq-Fwd primer | ACAGCGCTTCTCGGAAGAAA |
| | use for mutants screening - | |
| | sequencing | |
| 18 | Seipinseq-Rev primer | AGAAGGTAATATTCACTGCCTGTCA |
| | use for mutants screening - | |
| | sequencing | |

MUTANTS

| | | |
|---|---|---|
| 19 | ΔtSeipin 1 nucleic acid | ATGGACCGACGCGTTTCCCGACGCCGGTC |
| | sequence of the 1st exon | GCAAACGTCCCTAGTTGTGGCAGAAGAAG |
| | Annotations same as in the wild type | CGCACGCTG [symbol] CCGAGGACCCATCCGC |
| | sequence above | GTTGCAACGCATCGTTGAATACGCAGCGG |
| | In biggest character: the mutations | TGCGTGTGCTAGCCCGACAATTTCCCTCAA |
| | resulted from the CRISPR-Cas9 | GAAACGCACATGGTAGAAGCAGTACGGAA |
| | gene editing with the sgRNA1: | ATACCATTCAATCCATACCGAGAGCATATT |
| | addition of TG in the targeted | CCTGAGTTTGGAGGCGTTTTGCCATTTGTT |
| | sequence for guide RNA1 and | TTGCGATTGCTAAAAACAATACTTCTATCCG |
| | replacement of C by A in the first | GCTTACTT [symbol] TGACTGTATCACTAGCTTCGT |
| | transmembrane domain sequence. | ATTGGATGTGCTATCAAGCCGCCATGCCGT |
| | | CTCGGCACTCCAGTAAGCTGCTGTATTTCG |
| | The other mutations (in big | ACTATACAGGGTCTACGTTGCCACGGCTGA |
| | characters) are SNP known silent | TGGCAGCCAAAGTTCCATTTCGCCCCGTC |
| | mutations between our sequence | GAAGACAGCGAGTCCAGCTATCAAGGTCC |
| | and reference sequence | ATGGGCTCTGGTAGATTTGTTCTCTAAGCA |
| | (http://protists.ensembl.org/Phaeod | GCCGCAGTGGGAGGCATTCAGTCATGAAA |
| | actylum_tricornutum/Gene/Variation | TTCTACCGCCGCCTACCACATCGACGCGA |
| | _Gene/Table?db=core;g=Phatr3_J4 | CTTTTACAGCCCAAGCAGGACTACTACATT |
| | 7296;r=13:218930- | GAAATAGTGTTGGACTTACCGGAGTCGGAA |
| | 220524;t=Phatr3_J47296.t1) | CACAATCAGATGCTGGGCATGTTTGGTGTC |
| | | GTCACGGAACTCTATTCACGCAACGGAACC |
| | | AAACTTGCCTTATCTCGGCGGTCGATGCGC |
| | | ATACCGCATGAGAGCCACTGGATATCGGTT |
| | | GTGCGCAAGATGGCACTCTTGGCACCCCT |
| | | ATTGATTGGCGCCATCGAAGAAACACGAAC |
| | | GGTAACGGTGCCATCAT |
| 20 | ΔPtSeipin8 nucleic acid | ATGGACCGACGCGTTTCCCGACGCCGGTC |
| | sequence of the 1st exon | GCAAACGTCCCTAGTTGTGGCAGAAGAAG |
| | Annotations same as in the wild type | CGCACGCTGCCGAGGACCCATCCGCG [symbol] T |
| | sequence above | GCAACGCATCGTTGAATACGCAGCGGTGC |
| | | GTGTGCTAGCCCGACAATTTCCCTCAAGAA |
| | In biggest character: the mutations | ACGCACATGGTAGAAGCAGTACGGAAATA |
| | resulted from the CRISPR-Cas9 | CCATTC [symbol] AATCCATACCGAGAGCATATTC |
| | gene editing with the sgRNA8: | CTGAGTTTGGAGGCGTTTTGCCATTTGTTT |
| | replacement of T by a C and addition | TGCGATTGCTAAAAACAATACTTCTATCCG |
| | of a A in the targeted sequence of | GCTTACTTCTGACTGTATCACTAGCTTCGT |
| | guide RNA8 | ATTGGATGTGCTATCAAGCCGCCATGCCGT |
| | | CTCGGCACTCCAGTAAGCTGCTGTATTTCG |
| | The other mutations (in big | ACTATACAGGGTCTGCGTTGCCACGGCTG |
| | characters) are silent mutations | ATGGCAGCCAAAGTTCCATTTCTCCCCGTC |
| | between our sequence and | GAAGACAGCGAGTCCAGCTATCAAGGTCC |
| | reference sequence | ATGGGCTCTGGTAGATTTGTTCTCTAAGCA |
| | | GCCGCACTGGGAGGCATTCAATCATGAAAT |
| | | TCTACCGCCGCCTACCACATCGACGCGAC |
| | | TTTTACAGCCCAAGCAGGACTACTACATTG |
| | | AAATAGTGTTGGACTTACCGGAGTCGGAAC |
| | | ACAATCAGATGCTGGGCATGTTTGATGTCG |
| | | TCACGGAACTCTATTCACGCAACGGAACCA |
| | | AACT |
| 21 | ΔtSeipin 1 amino acid | MDRRVSRRRSQTSLVVAEEAHAVPRTHPRC |
| | sequence of the 1st exon | NASLNTQRCVC* |
| | The amino acids in italic | |
| | correspond to parts between | |
| | the frameshift and the end of | |
| | translation where the | |
| | translated sequence is | |
| | different from the WT | |
| | sequence. *: stop codon. | |
| 22 | ΔPtSeipin8 amino acid | MDRRVSRRRSQTSLVVAEEAHAAEDPSALQ |
| | sequence of the 1st exon | RIVEYAAVRVLARQFPSRNAHGRSSTEIPFKS |
| | The amino acids in italic | IPRAYS* |
| | correspond to parts between | |
| | the frameshift and the end of | |
| | translation where the | |
| | translated sequence is | |
| | different from the WT | |
| | sequence. *: stop codon. | |

REFERENCE SEQUENCE FOR PERCENTAGE IDENTITIES BETWEEN SEIPIN
PROTEINS

| | | |
|---|---|---|
| 23 | Pt Seipin reference sequence | IEIVLDLPESEHNQMLGMFGVVTELYSRNGT |
| | selected to calculate a % | KLALSRRSMRIPHESHWISVVRKMALLAPLLI |
| | identity of the Seipin in the | GAIE |
| | phylogenetic tree compared | |
| | to Pt Seipin, the said | |
| | reference sequence | |
| | comprising 7 amino acids of | |
| | third B-strand before the | |
| | PESxxN motif and going up | |
| | the 6st beta-strand, excluded | |
| | Bold character: Strongly conserved | |
| | motif PESxxN linking the third ß- | |
| | strand with a small a-helix | |
| 24 | Single guide sgRNA 1 | AGAAGAAGCGCACGCTGCCG |
| 25 | Single guide sgRNA 8 | TTCAATCCATACCGAGAGCA |
| 26 | WT Pt Seipin (DNA) | See figure 3 |
| 27 | ΔPtSeipin8 (DNA) | See figure 3 |
| 28 | ΔPtSeipin1 (DNA) | See figure 3 |
| 29 | Consensus sequence (DNA) | See figure 3 |
| 30 | WT Pt Seipin (protein) | See figure 4 |

### Seipin Proteins of Stramenopiles and Diatoms Microalgae

Lipid droplets (LDs) are endoplasmic reticulum (ER)-derived subcellular organelles dedicated for storing metabolic energy in the form of neutral lipids (NLs). The anhydrous core of these droplets is composed of the two most abundant NLs, triacylglycerol (TAG) and steryl esters. This oily drop is shielded from the aqueous environment by a monolayer of phospholipids, which harbor a set of LD-specific proteins, including lipases, acyltransferases and scaffolding proteins. The ER protein Seipin is key for LD biogenesis. Seipin forms a cage-like structure, with each seipin monomer containing a conserved hydrophobic helix and two transmembrane (TM) domains.

The Applicant made phylogenetic and structural analyses of Seipin proteins and detected some specificities shared by the Stramenopiles Seipin, and in particular diatom Seipin.

As illustrated in the examples and FIG. 1, diatom Seipin does not share a common ancestor with the plants and green algae Seipins, corroborating the unique features of this protein in diatoms.

Seipins are transmembrane proteins located in the ER membrane. They adopt a hairpin structure with two transmembrane alpha-helixes. The cytoplasmic N- and C-ter domains show very little conservation in terms of sequence and structures and their length is very variable. However, in spite of a general low sequence conservation of Seipin proteins, the secondary structure of the lumenal domain is remarkably conserved (FIG. 2A), as well as the tertiary structure (FIG. 2B).

Both transmembrane domains form α-helixes but do not show sequence conservation. The central part is mainly composed by 8 β-strands, forming a beta-sandwich. The hydrophobic α-helix (HH), located between the 5^{th} and the 6^{th} β-strands, plays an important role in Seipin oligomerization (Sui et al. 2018, Yan et al. 2018) as well as TAG clustering (Zoni et al., 2021). Another very small α-helix is found between the 3^{rd} and the 4^{th} β-strands in association with a very conserved small motif (PESxxN).

A finer analysis of the region between the first transmembrane domain and the very conserved PESxxN motif (FIG. 2A) reveals a particularity of all diatoms, with the presence of a long loop that often contains one or several α-helixes, surrounding the second β-strand (FIG. 2B).

We may consider 3 ways (criteria) to define the long loop:
1) if we consider the length of the domain located between the end of the 1st transmembrane domain and the conserved motif PESxxN (or PDSxxN), located at the end of the third β-strand (between amino acids positions P107 and L192 included, in the sequence of PtSeipin), this length must be greater than 80 amino acids;
2) if we consider the length of the domain located between two conserved motifs: FDY (or LDY), located just downstream of the 1st β-strand, and PESxxN (between amino acids positions T120 and L 192 included, in the sequence of PtSeipin), this length must be greater than 65 amino acids;
3) if we consider the length of the domain located between the last amino acid of the 1st β-strand and the first amino acid of the third β-strand constituting the beta-sandwich (between amino acids positions Y116 and D183 included, in the sequence of PtSeipin), this length must be greater than 60 amino acids.

So, according to the 1^{st} definition (criteria), the long loop will generally have a number of amino acids ranging from 80 to 160 amino acids, in particular from 84 to 154 amino acids.

By contrast, the length of the same region ranges between 50 and 75 amino acids in the tested land plants, 40 and 60 aminoacids in the tested Fungi and Animalia, and around 70-75 amino acids in Oomycota and Phaeophyceae.

So, in a particular embodiment, the wild type homologous Seipin gene of Stramenopiles microalgae of interest in the present invention encodes an amino acid sequence comprising (i) a long loop between the 1st and the third β-strands of the beta-sandwich of the lumenal domain, with a length of at least 80 amino acids located between the end of the 1st transmembrane domain and the conserved motif PESxxN (or PDSxxN), located at the end of the third β-strand, in particular between amino acids positions P107 and L 192 included, in the sequence of PtSeipin.

In particular, the long loop has a sequence having at least 80%, 90% or 100% of identity with the sequence SEQ ID NO:6.

In addition, the Applicant calculated the percentage identity of each amino acid Seipin sequence in the phylogenetic tree compared to Pt Seipin, wherein the alignment is based on a reference sequence Pt Seipin (SEQ ID NO:23) comprising 7 amino acids of third beta-strand before the PESxxN motif and going up the 6st beta-strand, excluded.

The percent identity of each diatom Seipin sequence is ranging from 41% to 64% identity with SEQ ID NO:23 (except for the Chaetoceros tenuissimus having 29.8% identity), whereas all other Seipin sequences aligned with the same SEQ ID NO: 23, all have a percent identity lower than 40%.

If we calculated an average percent identity for a group (ex: Diatoms), defined as the sum of the percentage identities of Seipin sequences with the sequence of reference (SEQ ID NO: 23), divided by the number of sequences considered in the said group, this average percentage identity is around 47% based on the 6 diatom Seipin sequences illustrated on FIG. 1 (without the Pt Seipin 100%), or 54% based on the 7 diatom Seipin sequences illustrated on FIG. 1 (with Pt Seipin 100%), whereas in SAR family, the compared average percentage identity with Pt Seipin is lower (38.7% or 43.1% respectively).

So, in a particular embodiment, the wild type homologous Seipin gene of diatom microalgae of main interest in the present invention encodes an amino acid sequence comprising (i) a long loop between the 1st and the third β-strands of the beta-sandwich of the lumenal domain, with a length of at least 80 amino acids located between the end of the 1st transmembrane domain and the conserved motif PESxxN (or PDSxxN), located at the end of the third β-strand, in particular between amino acids positions P107 and L 192 included, in the sequence of PtSeipin, and further comprising (ii) a sequence comprising the 7 amino acids before the PESxxN motif and going up to the 6st beta-strand (excluded), having a percentage identity of at least 40%, preferably at least 41% with the SEQ ID NO:23.

By at least 40% of identity, it means 40, 41, 42, 43, 44, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82? 83, 84, 85, 86, 87, 88, 89, 90, 91, 92, 93, 94, 95, 96, 97, 98, 99 or 100% identity with the SEQ ID NO:23.

As used herein, the “percentage identity” (or “% identity”) between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman, by means of the similarity search method of Pearson and Lipman (1988) or by means of computer software using these algorithms (GAP, BESTFIT, FASTA and TFASTA in the Wisconsin Genetics Software Package, Genetics Computer Group, 575 Science Dr., Madison, WI, or by the comparison software BLAST NR or BLAST P). The percentage identity between two nucleic acid or amino acid sequences is determined by comparing the two optimally-aligned sequences in which the nucleic acid or amino acid sequence to compare can have insertions or deletions compared to the reference sequence for optimal alignment between the two sequences. Percentage identity is calculated by determining the number of positions at which the amino acid, nucleotide or residue is identical between the two sequences, preferably between the two complete sequences, dividing the number of identical positions by the total number of positions in the alignment window and multiplying the result by 100 to obtain the percentage identity between the two sequences.

The Applicant also noted specific amino acid sequences or patterns conserved within diatoms Seipin and not conserved within Eustigmatophytes Seipin, in particular:
the amino acid sequence between the 5^{th} and 6^{th} β-strands including the alpha-hydrophobic helix that is well conserved between Diatoms Seipin sequences (SEQ ID NO: 7); the different species may have up to 3 not conserved amino acids (so the diatoms sequences have at least 85%, in particular at least 90% and even 100% identity with said SEQ ID NO: 7, with 100% identity with the pattern PHES (or PYES); and the amino acids E, S, K at position 4, 9 and 13 on this sequence of 28 amino acids (from left to right) are changed into R, R and S in Eustigmatophytes Seipin sequences;
amino acid pattern ‘PHES’ after the 5^{th} β-strand (SEQ ID NO: 8); and/or
amino acid pattern ‘IGKE’ in the 8^{th} β-strand (SEQ ID NO:9).

So, according to another particular embodiment, the wild type homologous Seipin gene of diatom microalgae of main interest in the present invention encodes an amino acid sequence comprising (i) a long loop between the 1st and the third β-strands of the beta-sandwich of the lumenal domain, with a length of at least 80 amino acids located between the end of the 1st transmembrane domain and the conserved motif PESxxN (or PDSxxN), located at the end of the third β-strand, in particular between amino acids positions P107 and L 192 included in the sequence of PtSeipin, and further comprising (iii) a sequence having at least 85% of identity with SEQ ID NO:7, located between the 5^{th} and 6^{th} β-strand including the α-hydrophobic helix, and preferably also the pattern PHES' after the 5^{th} β-strand (SEQ ID NO: 8) and/or the pattern ‘IGKE’ in the 8^{th} β-strand (SEQ ID NO:9).

By at least 85% of identity, it means 85, 86, 87, 88, 89, 90, 91, 92, 93, 94, 95, 96, 97, 98, 99, or 100% identity with the said sequences.

The sequence identity is defined as disclosed above.

So, in a particular and preferred embodiment, the wild type homologous Seipin gene of diatom microalgae of main interest in the present invention encodes an amino acid sequence comprising (i) a long loop between the 1st and the third β-strands of the beta-sandwich of the lumenal domain, with a length of at least 80 amino acids located between the end of the 1st transmembrane domain and the conserved motif PESxxN (or PDSxxN), located at the end of the third β-strand, in particular between amino acids positions P107 and L 192 included, in the sequence of PtSeipin, and advantageously further comprising (ii) a sequence comprising the 7 amino acids before the PESxxN motif and going up to the 6^{st} beta-strand (excluded), having a percentage identity of at least 40%, preferably at least 41% with the SEQ ID NO:23, and/or (iii) a sequence having at least 85% of identity with SEQ ID NO:7, located between the 5^{th} and 6^{th} β-strand including the α-hydrophobic helix, and preferably also the pattern PHES' after the 5^{th} β-strand (SEQ ID NO: 8) and/or the pattern ‘IGKE’ in the 8^{th} β-strand (SEQ ID NO:9).

In the case of substitution of one or more consecutive or non-consecutive amino acids (‘variants Pt Seipin’), substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding Seipin protein and of those specific examples defined below. Equivalent amino acids can be determined either on their structural homology with the amino acids for which they are substituted or on the results of comparative tests of biological activity between the various Seipin likely to be generated.

As a non-limiting example, Table 2 below summarises the possible substitutions likely to be carried out without resulting in a significant modification of the biological activity of the corresponding modified binding protein; inverse substitutions are naturally possible under the same conditions.

TABLE 2

| Original residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (E) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

Engineered Microalga with Loss of Function Seipin Gene

In a particular embodiment, loss of function of the homologous Seipin gene of Stramenopiles microalgae is obtained by genetic tools for silencing gene expression, in particular selected in the group consisting of mutation, RNA interference, antisens DNA, Knock-out gene, and small molecules inhibitors. Said Seipin gene, or one of its homologs, is described as being “silenced”.

In a particular and preferred embodiment, the loss of function is obtained by mutation of the targeted Seipin gene. A change in nucleotide sequence of the gene's coding region may lead to a different amino acid being added to the growing polypeptide chain, causing a change in protein structure and function. As example, when a mutation on the DNA strand creates a premature stop codon, the RNA template will not be completely translated, resulting in a protein with a lower molecular weight due to fewer amino acid residues. As a result, the truncated protein will also likely be nonfunctional.

Zinc-finger nucleases, nucleases, meganucleases (MNs), transcription activator-like effector nucleases (TALEN) and clustered regularly interspaced short palindromic repeats (CRISPR/Cas9) have emerged during the past decade as efficient tools for genome editing in many organisms. CRISPR-Cas9 is a simple two-component system that allows researchers to precisely edit any sequence in the genome of an organism. This is achieved by guide RNA, which recognizes the target sequence, and the CRISPR-associated endonuclease (Cas) that cuts the targeted sequence.

The PAM, also known as the protospacer adjacent motif, is about 2-6 nucleotides downstream of the DNA sequence targeted by the guide RNA and the Cas cuts 3-4 nucleotides upstream of it. The most commonly-used Cas9 from Streptococcus pyogenes recognizes the PAM sequence 5′-NGG-3′ (where “N” can be any nucleotide base).

So in a particular and preferred embodiment, the loss of function of the homologous Seipin gene is obtained preferably by mutation on the targeted Seipin gene, in particular by Zinc-finger nucleases, nucleases, meganucleases (MNs), transcription activator-like effector nucleases (TALEN) and clustered regularly interspaced short palindromic repeats (CRISPR/Cas9), preferably CRISPR-Cas9 or TALEN system, more preferably CRISPR-Cas9.

Genome editing in P. tricornutum was successfully demonstrated via TALEN (Mann et al., 2017) and CRISPR/Cas9 (Nymark et al., 2016). CRISPR/Cas9-mediated genome editing is a simple and versatile tool for creating targeted genome modifications.

The discovery of E. coli-mediated conjugation for the delivery of non-integrating extrachromosomal DNA (diatom episome) to the nucleus of P. tricornutum presented an ideal system for delivering CRIPSR/Cas9 for genome editing without its integration into the nuclear genome. Delivery of CRISPR/Cas9 has been achieved in P. tricornutum via the biolistic transformation of plasmids (Nymark et al., 2016) or RNP (Serif et al., 2018) and E. coli-mediated conjugation (Slattery et al., 2018). Of all these methods, E. coli-mediated conjugation requires no specialized equipment or chemicals to maintain auxotrophs (for RNP counterselection) and can be perfomed in a high throughput format.

Additionally, the diatom episome can be cured upon the removal of selection pressure which removes all exogenous genetic material.

In a particular and preferred embodiment, the mutated Seipin gene is obtained by CRISPR-Cas9-mediated gene editing on the targeted coding sequence.

The mutation on the targeted coding sequence generally leads to a frameshift introducing a stop codon early in the targeted coding sequence or leads to a deletion or insertion in this targeted coding sequence, preferably a frameshift introducing a stop codon early in the sequence.

As the two transmembrane domains and the sequence between these transmembrane domains are involved in the conformational structure and the function of Seipin protein, by targeting them, as for example targeting at least upstream or downstream of the 1^{st} transmembrane domain, will make at least one of these domains non-functional, resulting in a non-functional protein.

In particular, the targeting sequences are upstream or downstream of the 1^{st} transmembrane domain, preferably upstream.

By ‘upstream’ the 1^{st} transmembrane domain, it means for example at least 1, 5, 10 or 20 amino acids upstream the 1^{st} amino acid of the transmembrane domain. And the length of the targeted sequence to design the sgRNA generally ranges from 15 to 25 amino acids, generally 20 amino acids.

In a particular sequence, target sequences were selected with respect to their position in the PtSeipin (accession number Phatr3_J47296) gene to be as close as possible to the start codon of the gene.

In a particular and preferred embodiment, the mutated Seipin gene is obtained by CRISPR-Cas9-mediated gene editing by targeting one or two sequences upstream of the 1^{st} transmembrane domain, in particular one or two sequences having at least 80% identity, 90% identity or 100% identity with the sequences complementary to sgRNA 1 (AGAAGAAGCGCACGCTGCCG) and sgRNA 8 (TTCAATCCATACCGAGAGCA).

By at least 80% of identity, it means 80, 81, 82? 83, 84, 85, 86, 87, 88, 89, 90, 91, 92, 93, 94, 95, 96, 97, 98, 99 or 100% identity with the sequences complementary to sgRNA 1 (AGAAGAAGCGCACGCTGCCG) and sgRNA 8 (TTCAATCCATACCGAGAGCA).

The % identity is calculated as defined above.

### Vector

The present invention also relates to a vector comprising Cas9 sequence and one single guide sgRNA designed to target a sequence upstream or downstream of the 1^{st} transmembrane domain, in particular one single guide sRNA 1 (as shown in SEQ ID NO. 24: AGAAGAAGCGCACGCTGCCG) or sgRNA 8 (as shown in SEQ ID NO. 25: TTCAATCCATACCGAGAGCA).

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter (inducible or transient), genetic marker, antibiotic resistance, reporter gene, targeting sequence, protein purification tag. Vectors called expression vectors (expression constructs) specifically are for the expression of the transgene in the target cell, and generally have control sequences.

### Methods of Transformation

The methods for transforming microalgae are well known to a skilled person. For example, electroporation and/or chemical (such as calcium chloride- or lithium acetate-based) transformation methods or Agrobacterium tumefaciens-mediated transformation methods as known in the art can be used.

In a particular embodiment, the engineered unicellular microalga according to the invention is obtained by genetic transformation, in particular selected in the group consisting of biolistic transformation, electroporation and bacterial conjugation, preferably biolistic transformation (ex: plasmids on tungsten beads).

In a particular embodiment:
microalgae are maintained in exponential growth (maximum concentration 3·10⁶ cell·mL⁻¹) for a week prior to transformation;
for each transformation, plasmidic DNA were added to tungsten beads;
biolistics transformation was performed under a laminar flow hood with a Biolistic Particle Delivery System under recommendations of the supplier (ex: BioRad);

The engineered unicellular microalga is, according to a preferred embodiment, a diatom, such as the ones selected in the group consisting of Thalassiosira pseudonana, Thalassiosira oceanica, Chaetoceros tenuissimus, Fistulifera solaris, Phaeodactylum tricornutum, Nitzschia inconspicua, Fragilariopsis cylindrus, preferably Phaeodactylum tricornutum (strain CCMP2561), in particular a Pennate diatom selected in the group consisting of Fistulifera solaris, Phaeodactylum tricornutum, Nitzschia inconspicua, Fragilariopsis cylindrus, preferably Phaeodactylum tricornutum (strain CCMP2561).

So the present invention also relates to an in vitro method of silencing the expression of the homologous Seipin gene in an unicellular Stramenopile microalga, in particular in Phaeodactylum tricornutum (strain CCMP2561), comprising a step using genetic tools selected in the group consisting of mutation, RNA interference, antisens DNA, Knock-out gene, and small molecules inhibitors, preferably mutation on the targeted Seipin gene, in particular by Zinc-finger nucleases, nucleases, meganucleases (MNs), transcription activator-like effector nucleases (TALEN) and clustered regularly interspaced short palindromic repeats (CRISPR/Cas9), preferably CRISPR-Cas9 or TALEN system, more preferably CRISPR-Cas9 on a sequence upstream of the 1^{st} transmembrane domain.

In a particular embodiment, the invention concerns an in vitro method of producing an engineered unicellular Stramenopile microalga, in particular engineered unicellular Phaeodactylum tricornutum having an increased triacylglycerols (TAGs) content when compared with the wild-type microalga cell of the same type cultured in the same condition, comprising:
i) Preparing a CRISPR-Cas9 plasmid comprising the insertion of one single guide RNA (sgRNA1 or sgRNA8) targeting sequence upstream of the 1^{st} transmembrane domain in the coding sequence of homologous Ptseipin gene (Phatr3 J47296 Phaeodactylum tricornutum),
ii) Detecting and selecting the CRISPR-Cas9 plasmids having correct sgRNA insertions, in particular via PCR using the forward sgRNA primer (Seipin-g1-Fwd or Seipin-g8-Fwd) and the pCas9-U6-Rev primer,
iii) Optionally adding the CRISPR-Cas9 plasmids to tungsten beads,
iv) Genetically transforming Phaeodactylum tricornutum (strain CCMP2561) with the CRISPR-Cas9 plasmid, preferably by biolistic transformation,
v) Screening the transformants by PCR amplification of Seipin's first exon using primers Seipin-PCR-Fwd and Seipin-PCR-Rev and selecting the colonies carrying mutated amino acid sequence ΔPtSeipin1 (SEQ ID NO: 21) or a mutated amino acid sequence ΔPtSeipin8 (SEQ ID NO: 22).

### Mutants

The present invention also concerns the engineered unicellular microalga according to the invention, wherein it comprises a mutated amino acid sequence truncated upstream of the 1^{st} transmembrane domain, in particular a mutated amino acid sequence having at least 80%, 90% or 100% identity with ΔPtSeipin1 (SEQ ID NO: 21) or a mutated amino acid sequence having at least 80%, 90% or 100% identity with ΔPtSeipin8 (SEQ ID NO: 22).

### Microalgae Culture

The present invention also relates to a microalgae culture comprising an engineered unicellular Stramenopile microalga as defined above according to the invention, preferably an engineered unicellular Phaeodactylum tricornutum (from strain CCMP2561).

The culture medium and culture conditions are disclosed hereunder.

The present invention relates to methods providing an engineered Stramenopile microalga comprising a mutant of Pt Seipin and culturing said engineered microalga thereby allowing the production of TAGs.

In particular embodiments, the triacylglycerol content in said engineered microalga is at least 110% (corresponding to an increase of at least a factor 1.1) of the triacylglycerol content of a corresponding microalga which does not comprise said mutant of Pt Seipin gene.

### In Vitro Method of Producing Triacylglycerols (TAGS)

Another subject-matter of the invention is an in vitro method of producing triacylglycerols (TAG) comprising culturing an engineered Stramenopile microalga as defined above according to the invention or a microalgae culture as defined above in a culture medium to produce TAG, in particular in normal growth conditions or preferably in stress conditions selected from nutrient starvation and/or light stress conditions.

In a particular embodiment, the in vitro method further comprises a step of recovering the TAG from the engineered Stramenopile microalgae, the culture medium or the whole culture.

As example, the step of recovering TAG from the engineered microalgae comprises a pressure stress, such as a mechanical pressure, acoustic wave, shear flow or low speed centrifugation, or any other cell disruptions methods as disclosed in Lee et al. (2012).

In a particular embodiment, the step of recovering TAG from the engineered microalgae comprises a centrifugation at 3500 g and higher, for 10 minutes or longer.

In a particular embodiment, the quantity of TAGs per cell (or per liter of culture or per liter of culture per day) of the engineered Stramenopile microalga of the invention is increased by at least a factor 1.1 compared to the quantity of TAGs per cell (or per liter of culture or per liter of culture per day) of the wildtype microalga cultured in the same conditions.

In a particular method, the production of TAG by the engineered microalga of the invention is increased in normal growth conditions of at least a factor 1.1 compared to wild type microalga cells of the same type cultured in the same conditions, more specifically a factor 3 to 4 or higher.

The culture of the microalgae is generally carried out in chemically defined media. Some chemically defined culture media that can be used in the invention contain a carbon source, a nitrogen and/or phosphate source and minerals, salts and vitamins necessary to their growth. The person skilled in the art knows well the elements necessary to microalgae growth.

By ‘normal growth conditions’ of Stramenopile microalga in the present invention, it means a culture medium and conditions culture with several parameters, such as day/night cycle, oxygenation by spin and or gas supply, temperature, light, and culture medium supplemented with nutrients such as carbon, nitrogen, phosphate, iron, and vitamins.

In particular, the parameters will comprise:
Day/night cycle: 12 h/12 h to 16 h/8 h;
Oxygenation by spin or supply of gas by bubbling;
Light conditions: exposure to 75-80 μmol·photon·m²·s⁻¹
Temperature between 15° C. to 25° C., preferably 20° C.;
Culture medium supplemented with nitrogen, phosphate, iron, and vitamins.

In a preferred embodiment, the culture medium is Enhanced Artificial Sea Water (ESAW) comprising minerals, salts, vitamins, containing excess of nitrogen and phosphate source, such as the one used in the examples: 326.7 mM NaCl, 25 mM Na₂SO₄, 8.03 mM KCl, 0.725 mM KBr, 0.372 mM H₃BO₃, 0.0657 mM NaF, 47.18 mM MgCl₂-6H₂O, 9.134 mM CaCl₂-2H₂O, 0.082 mM SrCl₂-6H₂O, 8.17 μM Fe-EDTA, 8.3 μM Na₂EDTA-2H₂O, 0.254 μM ZnSO₄-7H₂O, 0.0672 μM CoCl₂-6H₂O, 2.73 μM MnCl₂-4H₂O, 6.12 nM Na₂MoO₄-2H₂O, 1 nM Na₂SeO₃, 6.27 nM NiCl₂-6H₂O, 0.039 nM CuSO₄-5H₂O, 4.09 nM vitamin B8, 0.738 nM vitamin B12, 0.593 μM vitamin B1, containing an excess of nitrogen and phosphate source (10N10P: 0.549 mM NaNO₃, and 0.022 mM NaH₂PO₄—H₂O).

To ensure proper growth, volumes of cultures during biomass amplification do not exceed a fifth of the capacities of Erlenmeyers and do not exceed a maximum concentration of 10 millions cells·mL⁻¹. Liquid cultures were kept in an incubator (Infors, HT Multitron Pro) at 20° C. with constant agitation (100 rpm) and with 12/12 cycles of light (75 μmol·photon·m²·s⁻¹)/dark. This culture condition is later referred to as control (CT) condition.

In another particular embodiment, the production of TAG by the engineered microalga of the invention is increased in light stress conditions of at least a factor 1.2 compared to wild type microalga cells of the same type cultured in the same conditions, more specifically a factor 10 to 15 or higher, in particular after 8 days of exposure to at least 100 μmol·photon·m² s⁻¹, in particular at least 150 μmol·photon·m² s⁻¹ and preferably at least 200 μmol·photon·m²·s⁻¹

By ‘light stress conditions’, it means an exposure to at least 100 μmol·photon·m²·s⁻¹, in particular ranging from 100 μmol·photon m² s⁻¹ to 1000 μmol·photon m² s⁻¹, preferably from 150 μmol·photon·m² s⁻¹ to 400 μmol·photon·m²·s⁻¹. In particular, the light stress conditions are an exposure to at least 150 μmol·photon·m² s⁻¹ and preferably at least 200 μmol·photon·m² s⁻¹ for few days, in particular for 8 days.

In another particular embodiment, the accumulation of TAG by the engineered microalga of the invention is accelerated under nutrient starvation, in particular phosphate starvation with an increase of at least a factor 1.2 compared to wild type microalga cells of the same type cultured in the same conditions, more specifically a factor 2 to 3 or higher, in particular in the first few days of culture, more specifically 3-4 days.

By ‘nutrient starvation’ or ‘nutrient deprivation’, it means nitrogen and/or phosphate starvation, in particular in the first few days of culture, more specifically 3-4 days.

Such nutrient starvation triggers the accumulation of storage lipids in the subcellular organelles, referred to as lipid droplets (LDs). The LD core of Phaeodactylum tricornutum is enriched in TAG and enclosed by a monolayer of specific membrane lipids.

So advantageously, the mutant Pt Seipin microalga is grown in phosphate-depleted medium.

### Uses of TAGS

The ability of engineered microalgae of the invention to accumulate TAG has triggered their exploitation as host for fatty acid production, e.g. for biofuel production, for chemical applications or in food industry, such as for the industrial production of omega-3 polyunsaturated fatty acids.

So the invention furthermore relates to the use of the engineered Stramenopile microalga as defined above, or the microalgae culture of the invention, or directly the TAGs produced by the in vitro method according to said invention, for biofuel production, in food industry (ex: as food supplements), in feed industry (ex: for fisheries), in green chemistry (ex: for the production of polymers), in pharmaceutical industry (ex: health supplements) or for the production of cosmetics (as emollients in formulations), in particular for biofuel production.

To this end, they are added in customary amounts to the foodstuffs, feedstuffs, cosmetics or pharmaceuticals.

In a particular embodiment, the engineered Stramenopile microalga as defined above, or the microalgae culture of the invention, or directly the TAGs produced by the in vitro method according to said invention are used for biofuel production.

The present invention will be now illustrated with the non-limitative examples.

### Examples

The microalga used in the examples is Phaeodactylum tricornutum (Pt1) Bohlin Strain 8.6 CCMP2561 (Culture Collection of Marine Phytoplankton, now known as NCMA: National Center for Marine Algae and Microbiota) was used in experiments.

### 1. Materials and Methods

### 1.1 Cloning

Single guide RNA (sgRNA) were designed using the phytoCRISP-Ex website (www.phytocrispex.biologie.ens.fr/CRISP-Ex/) and P. tricornutum's reference genome (accession number GCA_000150955). NGG was chosen as the Protospacer Adjacent Motif (PAM) sequence (PAM sequence 5′-NGG-3′). Targets were selected with respect to their position in the PtSeipin (accession number Phatr3_J47296) gene to be as close as possible to the start codon of the gene. An additional BLAST was performed on P. tricornutum genome using the preselected CRISPR targets sequence to insure that no similar sequences were found elsewhere in the genome. Primers pairs corresponding to the chosen sgRNA sequences (Table 1) were annealed by heating 2.5 μl of each primer at 100 μM in 45 μl of annealing buffer (1 mM EDTA, 50 mM NaCl, 10 mM Tris pH 7.6) at 95° C. for 4 minutes then allowing the mix to cool down at room temperature for 45 minutes.

The primers are listed in the table 1 disclosed above in the description.

The pKSdiaCAs9_sgRNA_ZeoR (Seydoux et al. 2022), as figured in FIG. 17, was digested by BsaI (New England BioLabs) for 1 h at 37° C. Annealing was performed with 1 μl of annealed sgRNA and 100 ng of linearized vector using T4 DNA Ligase (New England BioLabs). The ligation product was transformed in E. coli DH5α. PCR screening with Phire Plant Direct PCR Master Mix (F160, ThermoFisher Scientific) was performed on bacteria colonies to assess the presence of the insert using the forward sgRNA primer (Seipin-g1-Fwd or Seipin-g8-Fwd) and the pCas9-U6-Rev primer (Table 1).

Plasmids from positive colonies were extracted using the NucleoSpin Plasmid kit (Macherey-Nagel) and sent for sequencing to Macrogen (The Netherlands) using the M13-Reverse primer (Thermofisher). Plasmid midipreps (NucleoBond Xtra Midi kit, Macherey Nagel) were performed to obtain vectors with correct sgRNA insertions at a minimal concentration of 1 μg/μl.

### 1.2 Phylogenetic Analyses

The amino acid sequence of the putative PtSeipin protein (product of the locus Phatr3J_47296) was used as query to search all the sequence databases (NCBI, Ensembl, JGI) to retrieve the highest number of sequences suitable for phylogenetic analyses. The sequences were handled using BioEdit Sequence Alignment Editor computer program (Hall 1999). Multiple sequence alignment was performed using MAFFT (Katoh et al 2019) tool implemented in NGphylogeny.fr (Lemoine et al 2019). The phylogenetic analysis was performed in MEGA X using the Neighbor-Joining method (Saitou and Nei 1987). The optimal tree with the sum of branch length=48.43171304 is shown. The percentage of replicate trees in which the associated taxa clustered together in the bootstrap test (5000 replicates) are shown next to the branches. The tree is drawn to scale in substitutions per site. The evolutionary distances were computed using the JTT matrix-based method (Jones et al 1992). The rate variation among sites was modeled with a gamma distribution (shape parameter=2). This analysis involved 52 amino acid sequences and a total of 1511 positions (including gaps). All ambiguous positions were removed for each sequence pair (pairwise deletion option). The resulting tree was edited in MEGA X.

### 1.3 Structural Analysis

Modelisation of the PtSeipin protein was performed using AlphaFold (Varadi et al., 2021). The ChimeraX software (https://www.rbvi.ucsf.edu/chimerax) was used for visualization and analysis of the models. The PESxxN motif was identified using the MEME (Multiple Em for Motif Elicitation) motif discovery online software (https://meme-suite.org/meme/tools/meme), using as queries the sequences already used for phylogenetic analysis. Research parameters were set to detect 10 motifs ranging between 6 and 20 amino acids. The PESxxN containing motif was the first hit.

### 1.4 Culture of Microalgae

Phaeodactylum tricornutum (ecotype Pt1 strain CCMP2561) cells were cultured in Enhanced Artificial Sea Water (ESAW) (326.7 mM NaCl, 25 mM Na₂SO₄, 8.03 mM KCl, 0.725 mM KBr, 0.372 mM H₃BO₃, 0.0657 mM NaF, 47.18 mM MgCl₂-6H₂O, 9.134 mM CaCl₂-2H₂O, 0.082 mM SrCl₂-6H₂O, 8.17 μM Fe-EDTA, 8.3 μM Na₂EDTA-2H₂O, 0.254 μM ZnSO₄-7H₂O, 0.0672 μM CoCl₂-6H₂O, 2.73 μM MnCl₂-4H₂O, 6.12 nM Na₂MoO₄-2H₂O, 1 nM Na₂SeO₃, 6.27 nM NiCl₂-6H₂O, 0.039 nM CuSO₄-5H₂O, 4.09 nM vitamin B8, 0.738 nM vitamin B12, 0.593 μM vitamin B1), containing an excess of nitrogen and phosphate source (10N10P: 0.549 mM NaNO₃, and 0.022 mM NaH₂PO₄—H₂O). To ensure proper growth, volumes of cultures during biomass amplification do not exceed a fifth of the capacities of Erlenmeyers and do not exceed a maximum concentration of 10 millions cells·mL⁻¹. Liquid cultures were kept in an incubator (Infors, HT Multitron Pro) at 20° C. with constant agitation (100 rpm) and with 12/12 cycles of light (75 μmol·photon·m²·s⁻¹)/dark. This culture condition is later referred to as control (CT) condition.

For phosphate (—P) starvation, cells were collected from a CT culture in exponential phase, centrifuged at 1500 g for 10 minutes, washed 3 times and resuspended in ESAW without NaH₂PO₄—H₂O (10N00P). For higher light (HL) condition, cells were cultured in ESAW 10N10P under the temperature, agitation and light cycles described above but light intensity was increased to 200 μmol·photon·m²·s⁻¹. For all experiments, CT, HL and —P cultures were inoculated at an initial concentration of 10⁶ cells·mL⁻¹, while —N cultures were inoculated at 5.10⁶ cells·mL⁻¹.

To assess cell concentrations, absorbance at 730 nm (A730) was measured using a TECAN infinite M1000 pro microplate reader (TECAN, Austria) and the corresponding concentrations were calculated based on the following equation cell number=1.834.10⁻⁰⁸*A730+0.03758 (Conte et al., 2018). For all experiments, each sample (cell line/culture condition) was cultivated in triplicates and at each considered time point (days 4 and 8 for HL and —P, days 1 and 2 for-N and days 1 and 2 or 4 and 8 for CT), the samples were monitored using Nile Red staining (9-diethylamino-5-benzo[a]-phenoxazinone in 100% DMSO, Thermo fisher), confocal microscopy, transmission electronic microscopy and lipid content as presented below.

### 1.5 Microalgae Transformation

Microalgae were maintained in exponential growth (maximum concentration 3.10⁶ cell·m⁻¹) for a week prior to transformation. On the day preceding transformation, 100.10⁶ cells were collected by centrifugation at 1,000 g for 10 minutes at room temperature, and plated on ESAW 10N10P/1% Agar plates containing 0.237 μM Carbenicillin. The plates were placed overnight in a vertical incubator at 20° C. and with constant light.

For each transformation, 4-5 μg of plasmidic DNA, 50 μl of CaCl₂ 2.5M diluted in ethanol and 20 μL of spermidine 0.1 M (Sigma-Aldrich) were sequentially added to 3 mg of tungsten beads M17 (Bio-Rad, Hercules, USA) diluted in 50 μl glycerol 50% (v/v). The beads were thoroughly vortexed during all the process and the final mix was left to incubate for 1 minute at room temperature, then washed twice with cold 70% ethanol and resuspended in 50 μl absolute ethanol. Coated beads were kept under gentle vortex agitation until use.

Biolistics transformation was performed under a laminar flow hood with a Bio-Rad Biolistic PDS-1000/He Particle Delivery System (Bio-Rad) according to manufacturer recommendations and using 1550 psi rupture discs. For each transformation with a CRISPR-Cas9 construct, four successive shots were performed on the same plate with 90° rotations of the plates between each shot. The algae were left to recover for three days in a vertical incubator at 20° C. and with constant light before being transferred to a selection ESAW 10N10P/1% Agar plate containing 0.237 μM Carbenicillin and 0.07 μM Zeocin.

The first colonies started to appear after 4 to 6 weeks. When they got big enough, up to 20 colonies were labelled and resuspended in 10 μl ESAW. 5 μl were used for replating and 5 μl were kept for further analysis.

### 1.6 Conservation of the Strains

For long term storage, Phaeodactylum tricornutum cells are concentrated at 10 millions cells/mL in 15% DMSO then progressively freezed up to −80° C. (sequentially 1 hour at 4° C., 1 hour at −20° C.) and kept at −80° C.

For shorter term storage, Phaeodactylum tricornutum cells are kept on solid medium (ESAW10N10P, Agar 1%) containing 0.237 μM Carbenicillin alone (wild type: WT) or with 0.07 μM Zeocin (all mutant strains).

1.7 Screening of Transformants and their Purification

1 μl of cells suspended in ESAW was diluted in 9 μl Phire Dilution buffer (ThermoFisher Scientific) and heated at 95° C. for 10 minutes in order to favour cell breakage. PCR amplification of Seipin's first exon was then carried out using the Phire Plant Direct PCR Master Mix (F160, ThermoFisher Scientific) using primers Seipin-PCR-Fwd and Seipin-PCR-Rev (Table 1). PCR products were then purified using the Monarch® DNA Cleanup kit (NEB) and sent for sequencing to Macrogen with primer Seipinseq-Fwd and Seipinseq-Rev (Table 1). Analysis of sequences was performed using Tide (Tracking of Indels by Decomposition v3.3.0; http://shinyapps.datacurators.nl/tide/) and ICE (Inference of CRISPR Edits v2; https://ice.synthego.com/#/) online softwares. Colonies carrying pure deletions/insertions were kept as is, and mosaic colonies containing interesting mutations were further purified. For each mosaic colony, about 200 cells were plated on a new selection plate and algae were left to grow for 4-6 weeks. Up to 20 colonies were then picked up and screened using PCR/sequencing/sequence analysis as described above. The same process was repeated until 3 independent colonies containing 99-100% of interesting mutations were obtained for 2 of the guide RNAs.

### 1.8 Nile Red Staining

A 2.5 mg·mL⁻¹ stock solution of Nile Red (9-diethylamino-5-benzo[a]-phenoxazinone in 100% DMSO, Thermo fisher) is diluted 1:5 (v/v) in each aliquot of the culture.

### Confocal Microscopy

Images of microalgae stained with Nile Red were acquired using a Zeiss LSM880 FastAiryscan confocal microscope equipped with a 63×/1.4 oil-immersion Plan-Apochromat objective, running Zen 2.3 SP1 acquisition software. Nile red fluorescence was acquired with Argon laser (458, 488, 514 nm) excitation at 514 nm and emission detection between 580 and 640 nm, using the Airyscan mode. Brightfield images were acquired by differential interference contrast (DIC), using laser excitation at 488 nm and the photomultiplier tube detector for transmitted light (T-PMT).

### Harvesting Samples for Lipid Analysis

Samples for lipid analysis were collected at day 4 and 8. Cultures were centrifuged 10 minutes at 3500 g. Cell pellets were transferred into low-binding Eppendorf tubes, while supernatants were placed in 250 ml glass bottles (Schott, Germany). Both cells and supernatant were frozen in liquid nitrogen and kept at −80° C.

### Cells Lipid Extraction

Cells pellets stored at −80° C. were lyophilized overnight (CHRIST Alpha 2-4 LSCbasic) before lipid extraction. Frozen dried pellets were transferred into Corex glass tubes and ground, then of 4 mL of boiling ethanol was added to prevent the action of lipases. 2 mL of methanol followed by 8 mL of chloroform were then added at room temperature (RT) to extract the lipid phase and argon bubbling was performed for 1 minute in order to mix while preventing lipid oxidation. Tubes were then covered and left for one hour at RT. Filtration through glass wool was performed to eliminate cells debris and the filter was rinsed with 3 mL chloroform methanol (2:1, v/v). Biphase formation was initiated by adding 5 mL NaCl 1% (w/v) and centrifugating at 2500 g for 10 minutes. The lower organic phase was evaporated at 40° C. under argon flux. Lipids were resuspended in 1.5 mL chloroform, transferred into hemolysis tubes and the chloroform was evaporated under argon. Lipid fractions were stored at −20° C.

### Culture Medium Lipid Extraction

One volume of chloroform and one volume of methanol were directly added to the frozen supernatants which were left to thaw at room temperature. Once the samples completely thawed, they were transferred to a separating funnel and mixed by argon bubbling for 1 minute. Funnels were then capped and left for about 2 hours to allow phase separation. The lower organic phase was then collected and evaporated under argon. Lipids were resuspended in 3 ml of chloroform, transferred to hemolysis tubes and chloroform was again evaporated under argon. Lipid fractions were stored at −20° C.

### Methanolysis and Gas Chromatography Coupled to Flame Ionization Detection (GD-FID)

Methanolysis was performed using a MultiPurpose Sampler (MPS, Gerstel). Lipid fractions were left at room temperature for 15 minutes prior to sample preparation. They were then resuspended in 1 mL of chloroform of which 50 μL were taken to perform methanolysis. 5 μg of internal C15 standard (SIGMA) was added to each sample. 3 mL of methanolysis media (methanol:sulfuric acid 40:1, v/v) were added on the samples, which were then heated for 1 hour at 100° C. This reaction allows the separation of fatty acids from the glycerol backbone and the formation of fatty acid methyl esters (FAME). The reaction was stopped by addition of 3 mL water and 3 mL of hexane were subsequently added to extract the FAME. The upper phase was collected, evaporated under argon and FAME fractions can be stored at −20° C. or processed immediately through gas chromatography coupled to flame ionization detection (GC-FID). FAME were resuspended in 100 μL of hexane and loaded on a GC-FID Perkin Elmer Clarus 580 equipped with a 30-m long cyanopropyl polysilphenesiloxane column with a diameter of 0.22 mm and a film thickness of 0.25 μm for GC separation using nitrogen as a vector gas. Identification of the FAME was achieved by comparison of their retention time with those of standards (Sigma-Aldrich). Surface peak method using the internal standard (15:0 FA) allowed the FAME species quantification and determination of the glycerolipid concentration in the initial sample.

### Liquid Chromatography Coupled to Tandem Mass Spectrometry (LC-MS/MS)

25 nmol of total lipids were then used for identification by liquid chromatography coupled to tandem mass spectrometry (LC-MS/MS). Separation by LC was performed using an Agilent 1200 HPLC on a 5 μm diol column with a length of 150 mm and a diameter of 3 mm (Macherey-Nagel, Hoerdt, France). Tandem Mass spectrometry analysis was performed on a triple quadrupole 6460 (Agilent, Santa Clara, USA) using a Jetstream electrospray ion source. Glycerolipids identification and quantification were done using a MassHunter Workstation (Agilent, Santa Clara, USA), and quantities were adjusted through comparison with a quality control (QC) (Jouhet et al., 2017).

### 2. Result

2.1 the Seipins from Diatoms Form a Distinct Phylogenetic Group

The phylogenetic analyses show a tree composed of three main clades (FIG. 1), a basal clade containing green algae; a second clade containing plant Seipins split into two subclades (higher plant Seipin 1/lower plants and higher plant Seipin 2); a third clade containing stramenopiles, fungi and animals. The latter clade is split into four subclades: a fully supported clade clustering animals, sister to the fungal Seipins with a weak support (64%); a clade grouping several stramenopiles (e.g. Oomycetes, Eustigmatophytes and Pheophyceae); a fourth highly supported (97%) clade, robustly basal (97%) to the former three, clustering diatom Seipins only. At the best of our knowledge, the present phylogeny is the first including different kingdoms. It pinpoints the evolutionary origin of stramenopile Seipins from the non-photosynthetic host in the event of secondary endosymbiosis that gave rise to stramenopiles. Diatom Seipin does not share a common ancestor with the green lineage Seipins, corroborating the unique features of this protein in diatoms.

2.2 Seipins Show High Structure Conservation with Some Distinctive Features of Diatoms

Seipins are transmembrane proteins located in the endoplasmic reticulum (ER) membrane. They adopt a hairpin structure with two transmembrane alpha-helixes. The cytoplasmic N- and C-ter domains show very little conservation in terms of sequence and structures and their length is very variable. However, in spite of a general low sequence conservation of Seipin proteins, the secondary structure of the lumenal domain is remarkably conserved (FIG. 2A), as well as the tertiary structure (FIG. 2B). A finer analysis of the region between the first transmembrane domain and a very conserved PESxxN motif (FIG. 2A) reveals a particularity of all diatoms, with the presence of a long loop that often contains one or several α-helixes, (>80 amino acids) between the first and the third β-strand (FIG. 2B). The length of this loop varies between 84 and 154 amino acids in the tested species. By contrast, the length of the same region ranges between 50 and 75 amino acids in the tested land plants, 40 and 60 aminoacids in the tested Fungi and Animalia, and around 70-75 amino acids in Oomycota and Phaeophyceae. This particularity is also observed in the eustigmatophites Microchloropsis gaditana and Microchloropsis oceanica as well as in the lipogenic yeast Yarrowia lipolytica. But as illustrated by FIG. 2D, the amino acids in eustigmatophites do not have the amino acid pattern ‘PHES’ after the 5^{th} β-strand and the IGKE pattern in the 8^{th} β-strand, which is conserved in all diatoms sequences, and have less conservated hydrophobic α-helix in comparison to the diatoms sequences.

2.3 Mutations of Phaeodactylum tricornutum Seipin (PtSeipin) Leading to Truncated Proteins

PtSeipin mutants were generated using two different guides RNA with the CRISPR-Cas9 gene editing system. Genomic PtSeipin sequences of the mutants ΔPtSeipin1 (SEQ ID NO: 19) and ΔPtSeipin8 (SEQ ID NO: 20) are presented in FIG. 3 (incomplete sequences are figured).

Both mutants present a mutation of three nucleotides in 5′ of the PAM motif accordingly with the Cas9 cutting position. The resulting mutations are an insertion of two nucleotides (TG) for ΔPtSeipin1 and an insertion of one nucleotide (A) for ΔPtSeipin8.

In silico translation of the mutants sequences reveals that the sequence mutations lead to the introduction of premature stop codons and thus to truncated non-functional proteins ΔPtSeipin1 (SEQ ID NO: 21) and ΔPtSeipin8 (SEQ ID NO: 22) (FIG. 4).

2.4 Phaeodactylum tricornutum Seipin Knock Out Mutants Accumulates More Oil

Surprisingly, the loss of PtSeipin results in oil accumulation in the absence of any stress, while changes in cell growth remain very minor. This phenotype is enhanced in stress conditions: in particular, mild light stress results in huge oil accumulation with a very limited impact on cell growth. We compare two knock-out (KO) lines, ΔPtSeipin1 and ΔPtSeipin8, obtained by CRISPR-Cas9 using respectively guide RNA 1 and 8.

### 2.5 Oil Accumulation in Control Conditions

The WT and the two KO strains were followed in control conditions for 8 days. The KOs present a slightly slower growth compared to WT but all cell lines keep growing during this period (FIG. 5). The stationary phase is reached after approximately 7 days of culture.

Lipid Droplets are stained with Nile Red and observed by confocal microscopy. We observe Lipid Droplets in every cells but their size is increased in both KO lines at both observation times (Day 4 and Day8) (FIG. 6). In some mutant cells, the Lipid Droplets occupy the entire width of the cells, which is never observed in the WT under these growth conditions. Glycerolipid content analysis by GC-FID and LC-MS/MS shows that the total amount of lipids per cells is similar between the WT and KO lines (FIG. 7a). However, the glycerolipids profile is altered, with an important increase of Triacylglycerol (TAG) in the mutant lines, both in quantity (FIG. 7b) as well as percentage (FIG. 7c). These results suggest a redirection of the metabolic pathway to favor oil (TAG) production.

### 2.6 Oil Accumulation in Higher Light Stress

In order to assess the effect of high light stress on the mutants, the strains were placed in higher light condition (200 μmol·photon·m²·s⁻¹) for 8 days. Similarly to what was observed in control conditions, all lines keep growing even though the mutants appear slightly delayed (FIG. 8).

While lipid droplets accumulate in all strains after 4 days of higher light, their size is much increased in the KO lines and LD occupy an important part of the cell volume (FIG. 9). The shape of the LD is affected by the physical constraints of the cell and appears deformed for the bigger ones. After 8 days, the size and number of LD decreases in the WT but not in the KO strains.

Glycerolipid content analysis by GC-FID and LC-MS/MS shows that the total quantity of lipids per million cells slightly increases in the ΔPtSeipin8 line compared to the WT after 8 days of exposure to higher light (FIG. 10a). The amount of TAG is much increased (up to 16.3 fold) in both KO lines compared to the WT (FIG. 10b), and TAG in the mutant lines can represent up to 60% of total glycerolipids (FIG. 10c).

### 2.7 Oil Accumulation in Phosphate Starvation Stress

During phosphate starvation stress, cells keep growing for a few days as they remobilize their internal phosphate reserves, then stop growing when those reserves are depleted (FIG. 11). In this condition, there is no significant difference in growth between the WT and mutant lines.

Oil accumulation occurs faster in the mutant lines compared to the WT (FIG. 12). Phosphate deficiency is not yet completely installed in the WT after 4 days of culture while big Lipid droplets are already accumulating in the mutants.

Glycerolipid content analysis by GC-FID and LC-MS/MS shows that the total quantity of lipids in nmol per million cells in mutants are similar in the mutants and the WT after 4 days of cultures in a phosphate depleted medium (FIG. 13a). However, the TAG quantity is higher in the mutants compared to the WT (2 to 3-fold difference) (FIG. 13b) and TAG represent a higher percentage of total glycerolipids in the mutants, where they reach up to 40% of total glycerolipids (FIG. 13c).

### 2.8 Oil Liberation in the Medium of Culture

During microscopy survey of the cell lines in the different culture conditions, we observed Lipid Droplets floating in the medium of the KO cell lines. This was true for all conditions but appeared enhanced in the phosphate starvation condition (FIG. 14).

In order to quantify the liberation of TAG in the culture medium, we extracted lipids from the culture media in all conditions after 15 days of culture and analyzed the glycerolipids content by LC-MS/MS (FIG. 15). This analysis revealed first an increase of TAG in the culture medium in stress conditions compared to control and second a sharp increase in the amount of TAG in the culture medium of the mutants. The most important difference is observed in the higher light condition (up to 11.5 fold increase between WT and mutant ΔPtSeipin1) (FIG. 15b) but the liberation of TAG is more important in the phosphate starvation condition (100 fold change between CT and —P and up to 5.32 fold increase between WT and mutant ΔPtSeipin8) (FIG. 15c).

2.9 Lipid Droplets can be Released without Cell Destruction

It thus seems that mechanical shear stress induced by pipetting cells for microscopy or that gentle centrifugation used to pellet cells (2500 g for 10 minutes) and collect the culture media (3500 g for 10 minutes) are sufficient to trigger Lipid Droplets release in the medium. In one instance, we were able to observe the liberation of a Lipid Droplet from a cell during confocal imaging, where the pressure of the objective on the coverslip during a z-stack acquisition was sufficient to eject the Lipid Droplet from the cell (FIG. 16).

### REFERENCES

WO 2012/075543
Bowler, C. et al. (2008) ‘The Phaeodactylum genome reveals the evolutionary history of diatom genomes’, Nature, 456 (7219), pp. 239-244. Available at: https://doi.org/10.1038/nature07410.
Cai, Y. et al. (2015) ‘Arabidopsis SEIPIN Proteins Modulate Triacylglycerol Accumulation and Influence Lipid Droplet Proliferation’, The Plant Cell, 27(9), pp. 2616-2636. Available at: https://doi.org/10.1105/tpc. 15.00588.
Conte, M. et al. (2018) ‘Screening for Biologically Annotated Drugs That Trigger Triacylglycerol Accumulation in the Diatom Phaeodactylum’, Plant Physiology, 177(2), pp. 532-552. Available at: https://doi.org/10.1104/pp. 17.01804.
Fei, W., Du, X. and Yang, H. (2011) ‘Seipin, adipogenesis and lipid droplets’, Trends in endocrinology and metabolism: TEM, 22(6), pp. 204-210. Available at: https://doi.org/10.1016/j.tem.2011.02.004.
Guéguen, N., Le Moigne, D., Amato, A., Salvaing, J., & Maréchal, E. (2021). Lipid droplets in unicellular photosynthetic stramenopiles. Frontiers in Plant Science, 12, 639276.
Hu Q, Sommerfeld M, Jarvis E, et al. (2008) Microalgal triacylglycerols as feedstocks for biofuel production: perspectives and advances. Plant J. 2008; 54(4):621-639. doi:10.1111/j.1365-313X.2008.03492.
Jones, D. T., Taylor, W. R. and Thornton, J. M. (1992) ‘The rapid generation of mutation data matrices from protein sequences’, Computer applications in the biosciences: CABIOS, 8(3), pp. 275-282. Available at: https://doi.org/10.1093/bioinformatics/8.3.275.
Jouhet, J. et al. (2017) ‘LC-MS/MS versus TLC plus GC methods: Consistency of glycerolipid and fatty acid profiles in microalgae and higher plant cells and effect of a nitrogen starvation’, PloS One, 12(8), p. e0182423. Available at: https://doi.org/10.1371/journal.pone.0182423.
Kang, N. K. et al. (2022) ‘Microalgal metabolic engineering strategies for the production of fuels and chemicals’, Bioresource Technology, 345, p. 126529. Available at: https://doi.org/10.1016/j.biortech.2021.126529.
Katoh, K., Rozewicki, J. and Yamada, K. D. (2019) ‘MAFFT online service: multiple sequence alignment, interactive sequence choice and visualization’, Briefings in Bioinformatics, 20(4), pp. 1160-1166. Available at: https://doi.org/10.1093/bib/bbx108.
Kowthaman, C. N. et al. (2022) ‘A comprehensive insight from microalgae production process to characterization of biofuel for the sustainable energy’, Fuel, 310, p. 122320. Available at: https://doi.org/10.1016/j.fuel.2021.122320.
Lu, Y., Wang, X., Balamurugan, S., Yang, W. D., Liu, J. S., Dong, H. P., & Li, H. Y. (2017). Identification of a putative seipin ortholog involved in lipid accumulation in marine microalga Phaeodactylum tricornutum. Journal of applied phycology, 29, 2821-2829.
Lee, A. K., Lewis, D. M. and Ashman, P. J. (2012) ‘Disruption of microalgal cells for the extraction of lipids for biofuels: Processes and specific energy requirements’, Biomass and Bioenergy, 46, pp. 89-101. Available at: https://doi.org/10.1016/j.biombioe.2012.06.034.
Lemoine, F. et al. (2019) ‘NGPhylogeny.fr: new generation phylogenetic services for non-specialists’, Nucleic Acids Research, 47(W1), pp. W260-W265. Available at: https://doi.org/10.1093/nar/gkz303.
Mann, M. et al. (2017) ‘PtAUREO1a and PtAUREO1b knockout mutants of the diatom Phaeodactylum tricornutum are blocked in photoacclimation to blue light’, Journal of Plant Physiology, 217, pp. 44-48. Available at: https://doi.org/10.1016/j.jplph.2017.05.020.
Nymark, M. et al. (2017) ‘CRISPR/Cas9 Gene Editing in the Marine Diatom Phaeodactylum tricornutum’, Bio-Protocol, 7(15), p. e2442. Available at: https://doi.org/10.21769/BioProtoc.2442.
Saitou, N. and Nei, M. (1987) ‘The neighbor-joining method: a new method for reconstructing phylogenetic trees’, Molecular Biology and Evolution, 4(4), pp. 406-425. Available at: https://doi.org/10.1093/oxfordjournals.molbev.a040454.
Seydoux, C. et al. (2022) ‘Impaired photoprotection in Phaeodactylum tricornutum KEA3 mutants reveals the proton regulatory circuit of diatoms light acclimation’, New Phytologist, 234(2), pp. 578-591. Available at: https://doi.org/10.1111/nph.18003.
Slattery, S. S. et al. (2018) ‘An Expanded Plasmid-Based Genetic Toolbox Enables Cas9 Genome Editing and Stable Maintenance of Synthetic Pathways in Phaeodactylum tricornutum’, ACS Synthetic Biology, 7(2), pp. 328-338. Available at: https://doi.org/10.1021/acssynbio.7b00191.
Sui, X. et al. (2018) ‘Cryo-electron microscopy structure of the lipid droplet-formation protein seipin’, The Journal of Cell Biology, 217(12), pp. 4080-4091. Available at: https://doi.org/10.1083/jcb.201809067.
Taurino, M. et al. (2018) ‘SEIPIN Proteins Mediate Lipid Droplet Biogenesis to Promote Pollen Transmission and Reduce Seed Dormancy’, Plant Physiology, 176(2), pp. 1531-1546. Available at: https://doi.org/10.1104/pp. 17.01430.
Varadi, M. et al. (2022) ‘AlphaFold Protein Structure Database: massively expanding the structural coverage of protein-sequence space with high-accuracy models’, Nucleic Acids Research, 50(D1), pp. D439-D444. Available at: https://doi.org/10.1093/nar/gkab1061.
Yan, R. et al. (2018) ‘Human SEIPIN Binds Anionic Phospholipids’, Developmental Cell, 47(2), pp. 248-256.e4. Available at: https://doi.org/10.1016/j.devcel.2018.09.010.
Zoni, V. et al. (2021) ‘Seipin accumulates and traps diacylglycerols and triglycerides in its ring-like structure’, Proceedings of the National Academy of Sciences of the United States of America, 118(10), p. e2017205118. Available at: https://doi.org/10.1073/pnas.2017205118.

1-15. (canceled)

16. An engineered unicellular Stramenopile microalga comprising a loss of function of the homologous Seipin gene.

17. The engineered unicellular Stramenopile microalga according to claim 16, wherein the wild type homologous Seipin gene encodes an amino acid sequence comprising (i) a long loop between the 1st and the third β-strands of the beta-sandwich of the lumenal domain, with a length of at least 80 amino acids located between the end of the 1st transmembrane domain and the conserved motif PESxxN (or PDSxxN) located at the end of the third β-strand.

18. The engineered unicellular microalga according to claim 16, wherein the loss of function of the homologous Seipin gene is obtained by genetic tools for silencing gene expression, in particular selected in the group consisting of mutation, RNA interference, antisens DNA, Knock-out gene, and small molecules inhibitors.

19. The engineered unicellular microalga according to claim 18, wherein the mutated Seipin gene is obtained by CRISPR-Cas9-mediated gene editing on the targeted coding sequence.

20. The engineered unicellular microalga according to claim 19, wherein the mutated Seipin gene is obtained by CRISPR-Cas9-mediated gene editing by targeting one or two sequences upstream of the 1^{st} transmembrane domain.

21. The engineered unicellular microalga according to claim 16, obtained by genetic transformation, in particular selected in the group consisting of biolistic transformation, electroporation and bacterial conjugation.

22. The engineered unicellular microalga according to claim 16, wherein the Stramenopile microalga is a diatom, such as the ones selected in the group consisting of Thalassiosira pseudonana, Thalassiosira oceanica, Chaetoceros tenuissimus, Fistulifera solaris, Phaeodactylum tricornutum, Nitzschia inconspicua, Fragilariopsis cylindrus.

23. The engineered unicellular microalga according to claim 16, wherein it comprises a mutated amino acid sequence truncated upstream of the 1^{st} transmembrane domain.

24. A microalgae culture comprising an engineered unicellular Stramenopile microalga according to claim 16.

25. A vector comprising Cas9 sequence and one single guide sgRNA designed to target a sequence upstream or downstream of the 1^{st} transmembrane domain.

26. An in vitro method of producing triacylglycerols (TAG) comprising culturing an engineered microalga according to claim 16 in a culture medium to produce TAG.

27. The in vitro method according to claim 26, wherein the production of TAG is increased in normal growth conditions of at least a factor 1.1 compared to wild type microalga cells of the same type in the same conditions.

28. The in vitro method according to claim 26, wherein the production of TAG is increased in light stress conditions of at least a factor 1.2 compared to wild type microalga cells of the same type in the same conditions, more specifically a factor 10 to 15 or higher.

29. The in vitro method according to claim 26, wherein the accumulation of TAG is accelerated under nutrient starvation, with an increase of at least a factor 1.2 compared to wild type microalga cells of the same type in the same conditions.

30. The engineered unicellular microalga according to claim 16, wherein the long loop is between amino acids positions P107 and L192 included in the sequence of Pt Seipin.

31. The engineered unicellular microalga according to claim 16, further comprising (ii) a sequence comprising the 7 amino acids before the PESxxN motif and going up to the 6st beta-strand (excluded), having a percentage identity of at least 40% with the SEQ ID NO:23, and/or (iii) a sequence having at least 85% of identity with SEQ ID NO:7, located between the 5^{th} and 6^{th} beta-strands including the alpha-hydrophobic helix.

32. The engineered unicellular microalga according to claim 16, wherein the loss of function of the homologous Seipin gene is obtained by Zinc-finger nucleases, nucleases, meganucleases (MNs), transcription activator-like effector nucleases (TALEN) and clustered regularly interspaced short palindromic repeats (CRISPR/Cas9).

33. The engineered unicellular microalga according to claim 19, wherein the mutated Seipin gene is obtained by CRISPR-Cas9-mediated gene editing on the targeted coding sequence by targeting sequences upstream or downstream of the 1^{st} transmembrane domain.

34. The engineered unicellular microalga according to claim 19, wherein the mutated Seipin gene is obtained by CRISPR-Cas9-mediated gene editing by targeting one or two sequences upstream of the 1^{st} transmembrane domain, said one or two sequences having at least 80% identity, 90% identity or 100% identity with the two sequences complementary to sgRNA 1 (SEQ ID NO. 24) and sgRNA 8 (SEQ ID NO. 25).

35. The engineered unicellular microalga according to claim 16, wherein the Stramenopile microalga is Phaeodactylum tricornutum (strain CCMP2561).
